(19) United States
(12) Patent Application Publication
(10) Pub. No.: US 2025/0019367 A1
(43) Pub. Date: Jan. 16, 2025

(54) Methods for Separation of Enantiomers

(71) Applicant: Mirati Therapeutics, Inc., San Diego, CA (US)

(72) Inventors: Michal Achmatowicz, San Diego, CA (US); Svitlana Kulyk, San Diego, CA (US); David Snead, San Diego, CA (US); Cheng Chen, San Diego, CA (US); Matthew Marx, San Diego, CA (US); Christopher Smith, San Diego, CA (US)

(21) Appl. No.: 18/697,948

(22) PCT Filed: Oct. 6, 2022
(86) PCT No.: PCT/US2022/045898

§ 371 Date: Apr. 2, 2024

Related U.S. Application Data
(60) Provisional application No. 63/252,973, filed on Oct. 6, 2021.
(60) Provisional application No. 63/352,504, filed on Jun. 15, 2022.

(51) Int. Cl. C07D 403/04; B01D 9/00; B01D 15/38; C07B 57/00

(52) U.S. Cl. CPC C07D 403/04; B01D 9/005; B01D 15/3833; C07B 57/00; B01D 2009/0086

(57) ABSTRACT

The present invention relates to methods for separating enantiomers of 5-phenyl and 5-naphthyl substituted 4-(aminomethyl)-6-(1-methyl-1H-pyrazol-4-yl)phthalazin-1(2H)-ones using N-Boc-L-phenylalanine, N-Boc-D-phenylalanine, and similar chiral acids.

### CROSS-REFERENCE TO RELATED APPLICATIONS

This application claims priority from U.S. Provisional Application No. 63/252,973, filed Oct. 6, 2021, and U.S. Provisional Application No. 63/352,504, filed Jun. 15, 2022, the disclosure of each of which is hereby incorporated by reference in its entirety.

### FIELD OF THE INVENTION

This invention relates to methods for separating enantiomers of 5-phenyl and 5-naphthyl substituted 4-(aminomethyl)-6-(1-methyl-1H-pyrazol-4-yl)phthalazin-1(2H)-ones using N-Boc-L-phenylalanine, N-Boc-D-phenylalanine, and similar chiral acids.

### BACKGROUND OF THE INVENTION

Certain 5-substituted 4-(aminomethyl)-6-(1-methyl-1H-pyrazol-4-yl)phthalazin-1(2H)-ones, such as Compounds 1 and 2 below, are axially chiral and therefore exist as atropisomers. Compound 1, axially chiral 2-(4-(4-(aminomethyl)-1-oxo-1,2-dihydrophthalazin-6-yl)-1-methyl-1H-pyrazol-5-yl)-3-fluoro-3,4-dihydronaphthalene-1-carbonitrile, and Compound 2, axially chiral 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile, are potent and selective inhibitors of PRMT5.

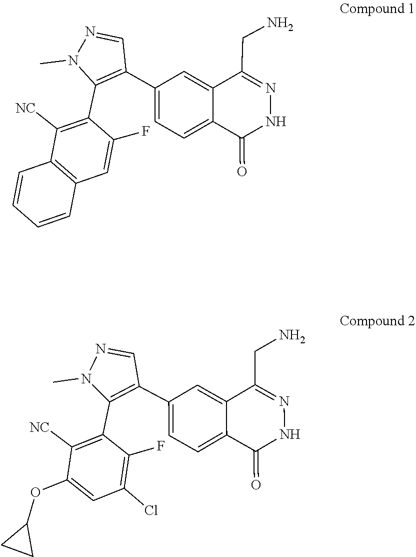 [SMILES: Cn1ncc(-c2ccc3c(=O)[nH]nc(CN)c3c2)c1-c1c(F)cc2ccccc2c1C#N ; Cn1ncc(-c2ccc3c(=O)[nH]nc(CN)c3c2)c1-c1c(F)c(Cl)cc(OC2CC2)c1C#N]

However, only the M-atropisomer of Compound 2 (which is depicted below and can be named (2A)-2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile (hereinafter Compound M-2)), is pharmacologically active.

The axial chirality in Compound 1 and Compound 2 is a consequence of restricted rotation between the phenyl moiety (Ring A) and the central N-methylpyrazole (Ring B). Molecules sharing this Ring A/Ring B motif, including Compound 1 and Compound 2 and their upstream intermediates, therefore exist as resolvable enantiomeric mixtures.

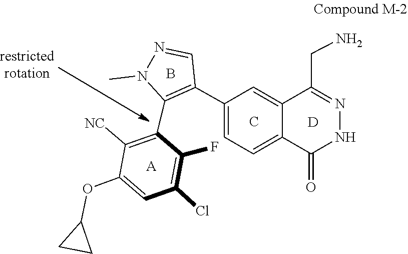

The pharmacologically active M-enantiomer of Compound 2 can be obtained via chiral chromatography. This compound has a specific rotation [α]D=35° (c=0.3, MeOH) when determined at 25° C. using material with 97.3% enantiomeric excess of (M) enantiomer.

A synthesis resulting in racemic Compound 2 is described in Published International Application WO2021/050915A1 (published Mar. 18, 2021, incorporated by reference herein). See racemic compound 4-230 at page 243; coupling method 4D at pages 195-196, and purification method 4-6 at page 198. The M- and P-enantiomers in racemic Compound 2 so synthesized were separated as described in WO2021/050915A1 in Examples 16-7 and 16-8 at page 307. This process of chiral chromatographic separation is disadvantageous because it is solvent intensive, non-scalable and expensive.

In view of the disadvantages outlined above, alternative approaches to the synthesis of pure or highly enriched M-enantiomers of Compound 1 and Compound 2 were sought.

An enantioselective variant of the Suzuki-Miyaura cross-coupling reaction was considered, but was deemed to not be feasible due to at least two major challenges: (1) heavily ortho-substituted building-blocks required for Suzuki-Miyaura reactions are difficult to cross-couple even in a racemic fashion, and (2) elevated temperature required for Suzuki-Miyaura reactions is incompatible with Compound 2 due to accelerated racemization under these conditions.

Because the known methods of synthesizing the M-enantiomer of Compound 2 were neither efficient nor scalable, and because theoretically alternate approaches such as the enantioselective variation of the Suzuki-Miyaura cross-coupling reaction were ruled out as unfeasible, improved, efficient methods for obtaining the M-enantiomer of Compound 2, i.e., Compound M-2, are needed.

### SUMMARY OF THE INVENTION

The invention includes the resolution of racemic Compound 2 using a chiral acid according to the following scheme:

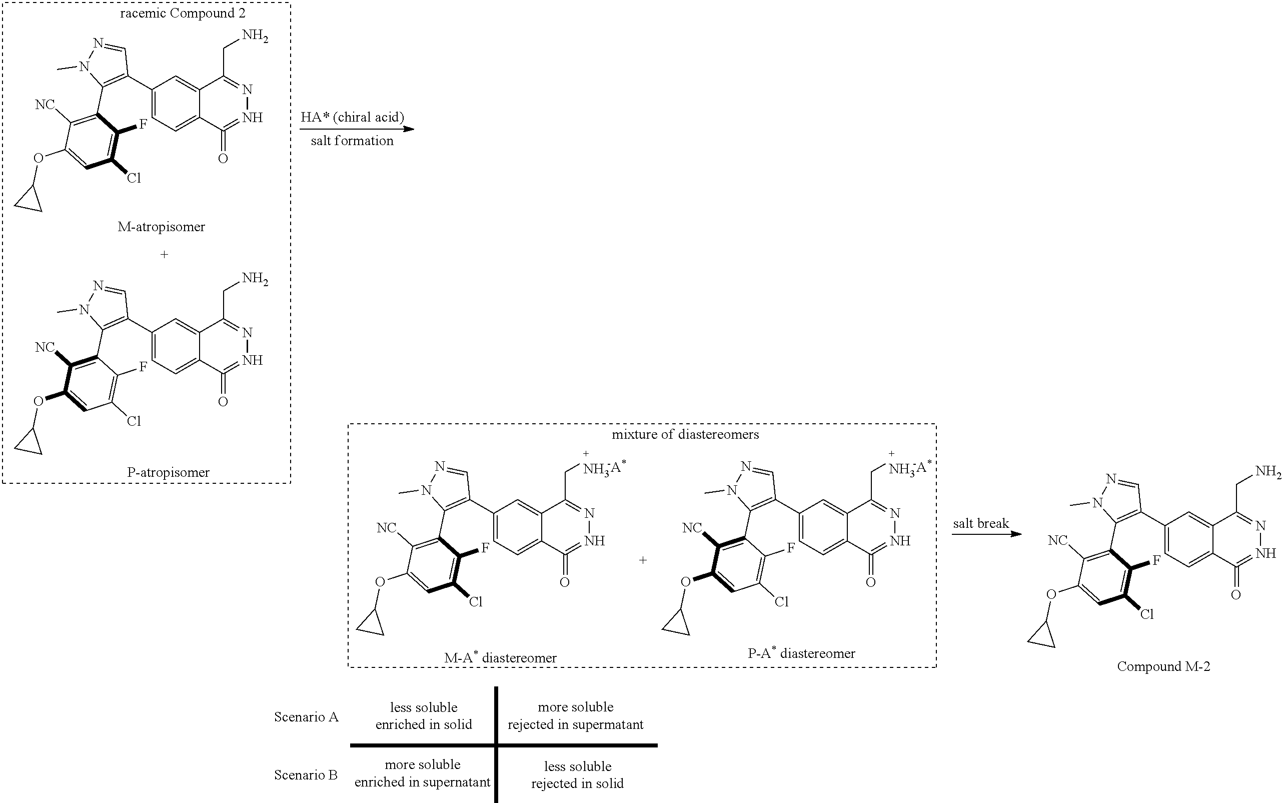

Although numerous chiral acids were tested, the inventors initially failed to identify a salt that would sufficiently differentiate between the M- and P-enantiomers of Compounds 1 and 2. It was then unexpectedly discovered that salt formation between the M- and P-enantiomer components of racemic Compound 1 and Compound 2 freebases and certain chiral acids (HA*), specifically N-Boc-L-phenylalanine and N-Boc-D-phenylalanine, results in two diastereomeric salts (M-A* and P-A*). In contrast to individual enantiomers, the resulting salts of Compound 1 and Compound 2 surprisingly possess different physicochemical properties, for instance different solubilities or crystallinities. After discovering that the chiral acids HA* (N-Boc-L-phenylalanine and/or N-Boc-D-phenylalanine) were suitable, it was determined that resolution of the racemates could proceed by either of two scenarios: In Scenario A the desired Compound 2 salt enantiomer (M-A*) is less soluble and therefore preferentially crystallizes, providing solids enriched in M-A* while undesired Compound 2 salt diastereomer (P-A*) is rejected in the supernatant. In Scenario B the undesired Compound 2 salt diastereomer (P-A*) is less soluble and preferentially crystallizes, consequently enriching the supernatant in Compound 2 salt M-A*.

The invention also provides salts of Compound 1 and Compound 2. In particular, the invention provides
the N-Boc-D-phenylalanine salt of (2M)-2-(4-(4-(aminomethyl)-1-oxo-1,2-dihydrophthalazin-6-yl)-1-methyl-1H-pyrazol-5-yl)-3-fluoro-3,4-dihydronaphthalene-1-carbonitrile;
the N-Boc-D-phenylalanine salt of (2P)-2-(4-(4-(aminomethyl)-1-oxo-1,2-dihydrophthalazin-6-yl)-1-methyl-1H-pyrazol-5-yl)-3-fluoro-3,4-dihydronaphthalene-1-carbonitrile;
the N-Boc-L-phenylalanine salt of (2M)-2-(4-(4-(aminomethyl)-1-oxo-1,2-dihydrophthalazin-6-yl)-1-methyl-1H-pyrazol-5-yl)-3-fluoro-3,4-dihydronaphthalene-1-carbonitrile;
the N-Boc-L-phenylalanine salt of (2P)-2-(4-(4-(aminomethyl)-1-oxo-1,2-dihydrophthalazin-6-yl)-1-methyl-1H-pyrazol-5-yl)-3-fluoro-3,4-dihydronaphthalene-1-carbonitrile;
the N-Boc-D-phenylalanine salt of (2M)-2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile;
the N-Boc-D-phenylalanine salt of (2P)-2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile
the N-Boc-L-phenylalanine salt of (2M)-2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile; and
the N-Boc-L-phenylalanine salt of (2P)-2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile.

The invention further also encompasses solid forms of the above described Boc-phenylalanine salts, in particular a crystalline form of N-Boc-D-phenylalanine salt of (2M)-2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile.

The invention also encompasses crystalline forms of (2Mf)-2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile and (2Mf)-2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloric acid salt. More specifically, the invention provides crystalline Form A of (2M)-2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile, i.e., crystalline Form A of Compound M-2. The invention also provides crystalline Form A and crystalline Form B of (2M)-2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloric acid salt.

The invention also provides systems for producing and isolating crystalline forms of (2M)-2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile and (2M)-2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloric acid salt, in particular, crystalline Form A of (2M)-2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile.

### BRIEF DESCRIPTION OF THE FIGURES

FIG. 1 is a flow chart showing the separation of highly enriched or pure Compound M-2 as a solid as described in Examples 1A and 1B.

FIG. 2 is a flow chart showing the separation of highly enriched or pure Compound M-2 in the liquid phase as described in Example 2.

FIG. 3 is a diagram of a system according to the invention that comprises a crystallization module and an epimerization module.

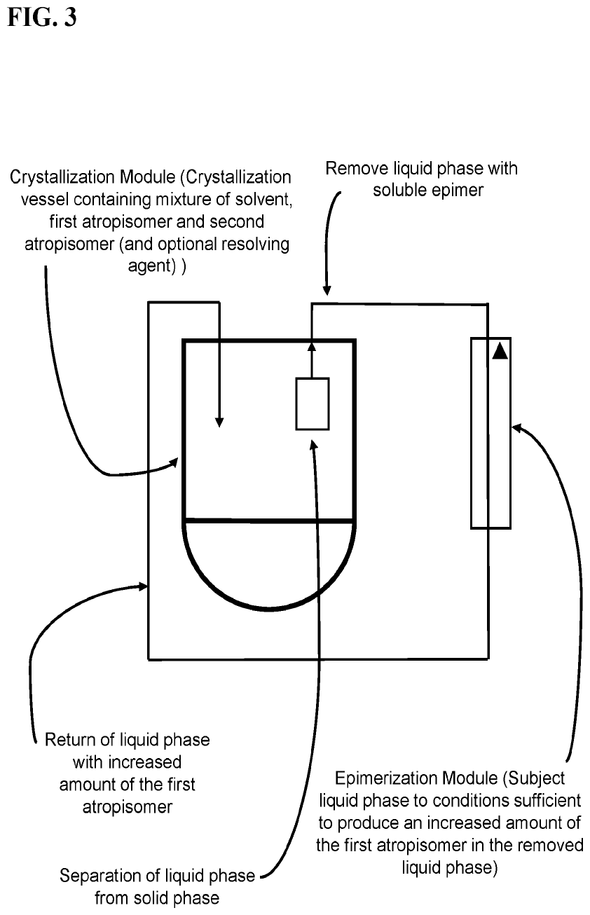

FIG. 4 is a 1H qNMR analysis according to an example embodiment.

FIG. 5 is an HPLC chromatogram of R-BINOL according to an example embodiment.

FIG. 6 is an HPLC chromatogram of S-BINOL according to an example embodiment.

FIG. 7 is an HPLC chromatogram of Supernatant after 1 hr of aging according to an example embodiment.

FIG. 8 is an HPLC chromatogram of eluent from flash epimerization at t=0 according to an example embodiment.

FIG. 9 is an HPLC of crystallized R-BINOL according to an example embodiment.

FIG. 10 is ¹H NMR in d₆-DMSO of Compound M-2 Boc-D-phenylalanine according to an example embodiment.

FIG. 11 is a chart demonstrating the fast equilibrium achieved using MSMPR-SPACE combination.

FIG. 12 is a diagram of a system according to the invention that comprises a crystallization module (MSMPR), a collection module or tank, and a racemization or epimerization module. In this figure, [symbol] signifies M-enantiomer in solution, [symbol] signifies P-enantiomer in solution, [symbol] signifies M-enantiomer in crystal-phase, [symbol] signifies the application of heat, and [symbol] signifies the application of cooling.

FIG. 13 is a chart demonstrating the distribution ratio in solid (Dₛ) according to an example embodiment.

### DETAILED DESCRIPTION OF THE INVENTION

As used herein, the term “atropisomer” refers to stereoisomers that exist as a consequence of hindered rotation about a single bond. In such compounds, energy differences due to steric strain or other contributors create a barrier to rotation sufficiently high to allow for isolation of individual conformers.

As used herein, Compound M-1 refers to (2M)-2-(4-(4-(aminomethyl)-1-oxo-1,2-dihydrophthalazin-6-yl)-1-methyl-1H-pyrazol-5-yl)-3-fluoro-3,4-dihydronaphthalene-1-carbonitrile, i.e., a compound having the following structure

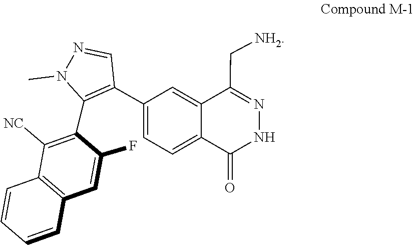

As used herein, Compound P-1 refers to (2P)-2-(4-(4-(aminomethyl)-1-oxo-1,2-dihydrophthalazin-6-yl)-1-methyl-1H-pyrazol-5-yl)-3-fluoro-3,4-dihydronaphthalene-1-carbonitrile, i.e., a compound having the following structure

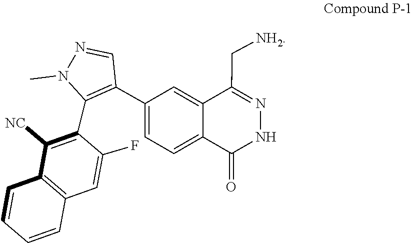

As used herein, Compound M-2 refers to (2M)-2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile, i.e., a compound having the following structure

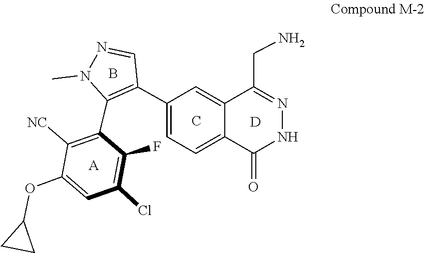

As used herein, Compound P-2 refers to (2P)-2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile, i.e., a compound having the following structure

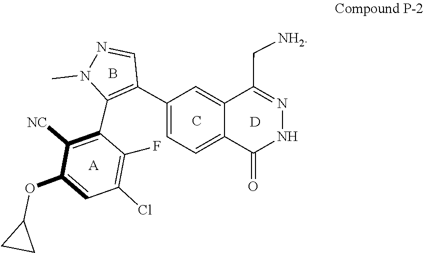

Identifying an appropriate chiral acid (HA*) capable of providing a sufficiently large discrimination (in terms of physical or chemical properties) between the M- and P-enantiomers of Compound 1 and Compound 2 was challenging, likely due to spatial separation between the axis of chirality and the chiral counterion, shown below for Compound M-2:

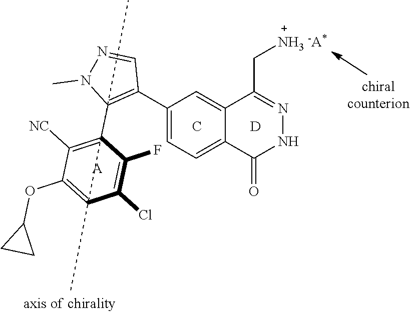

Extensive testing of numerous chiral acids with Compound 1 and Compound 2 resulted in minimal upgrade in either solids or supernatants. For instance, see Table 1, where the enantiomeric composition of Compound 2 for solids is shown to be in the 40%-60% to 60%-40% range for the majority of acids tested. However, it was unexpectedly discovered that certain chiral acids provided promising differentiation. For example, in certain embodiments, N-Boc-L-phenylalanine provided differentiation of 79%-21%, and N-Boc-D-phenylalanine provided differentiation of 7%-93% and 2%-98%, between the P and M enantiomers of Compound 2, depending on solvent (see Table 1, bottom).

TABLE 1
Enantiomeric composition of precipitates obtained
in the chiral acid screen of Compound 2

| | | Compound | Compound |
|---|---|---|---|
| Chiral Acid (HA*) | Solvent | P-2 (%) | M-2 (%) |
| di-p-toluoyl-L-tartaric acid | MeOH/DCM | 57 | 43 |
| | THF | 49 | 51 |
| di-p-toluoyl-D-tartaric acid | MeOH/DCM | 38 | 62 |
| | EtOH | 59 | 41 |
| | THF | 44 | 56 |
| | MeCN | 50 | 50 |
| (S)-10-camphorsulfonic acid | MeOH/DCM | 50 | 50 |
| | MeOH | 51 | 49 |
| | Anisole | 50 | 50 |
| | THF | 50 | 50 |
| | EtOAc | 50 | 50 |
| | MeCN | 50 | 50 |
| (R)-10-camphorsulfonic acid | MeOH/DCM | 50 | 50 |
| | THF | 50 | 50 |
| | MeOH | 49 | 51 |
| (S)-mandelic acid | MeOH/DCM | 49 | 51 |
| | THF | 49 | 51 |
| (R)-mandelic acid | MeOH/DCM | 53 | 47 |
| | EtOH | 54 | 46 |
| | THF | 47 | 53 |
| | EtOAc | 47 | 53 |
| | MeCN | 56 | 44 |
| dibenzoyl-L-tartaric acid | MeOH/DCM | 50 | 50 |
| | MeOH | 42 | 58 |
| | EtOH | 57 | 43 |
| | Anisole | 51 | 49 |
| | THF | 59 | 41 |
| | EtOAc | 50 | 50 |
| | MeCN | 50 | 50 |
| dibenzoyl-D-tartaric acid | MeOH | 59 | 41 |
| (R)-3,3,3-trifluoro-2-methoxy-2- | EtOH | 50 | 50 |
| phenylpropanoic acid | Anisole | 51 | 49 |
| | THF | 52 | 48 |
| | EtOAc | 56 | 44 |
| | MeCN | 50 | 50 |
| L-tartaric acid | EtOH | 50 | 50 |
| | Anisole | 50 | 50 |
| | THF | 50 | 50 |
| | EtOAc | 50 | 50 |
| | MeCN | 50 | 50 |
| N-acetyl-L-leucine | EtOH | 50 | 50 |
| | Anisole | 50 | 50 |
| | THF | 50 | 50 |
| | EtOAc | 50 | 50 |
| | MeCN | 51 | 49 |
| N-Boc-L-valine | THF | 47 | 53 |
| N-Boc-L-alanine | THF | 47 | 53 |
| N-Boc-L-proline | THF | 47 | 53 |
| (2S)-2-(6-methoxy-2-naphthyl)propanoic acid | THF | 47 | 53 |
| (2S)-2-(benzyloxycarbonylamino)-2- | THF | 47 | 53 |
| phenylacetic acid | | | |
| N-Boc-L-phenylalanine | THF | 79 | 21 |
| N-Boc-D-phenylalanine | THF | 7 | 93 |
| | MeOH | 2 | 98 |

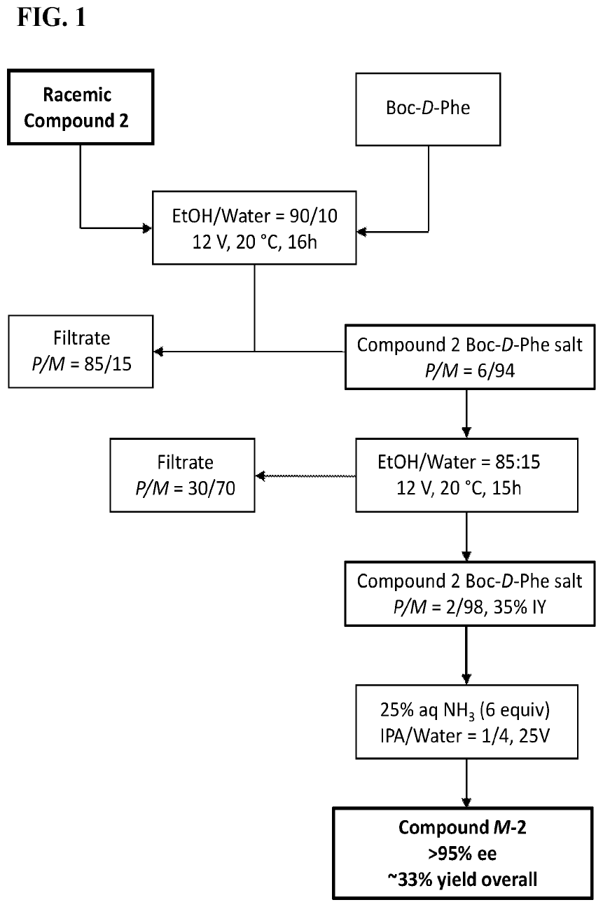

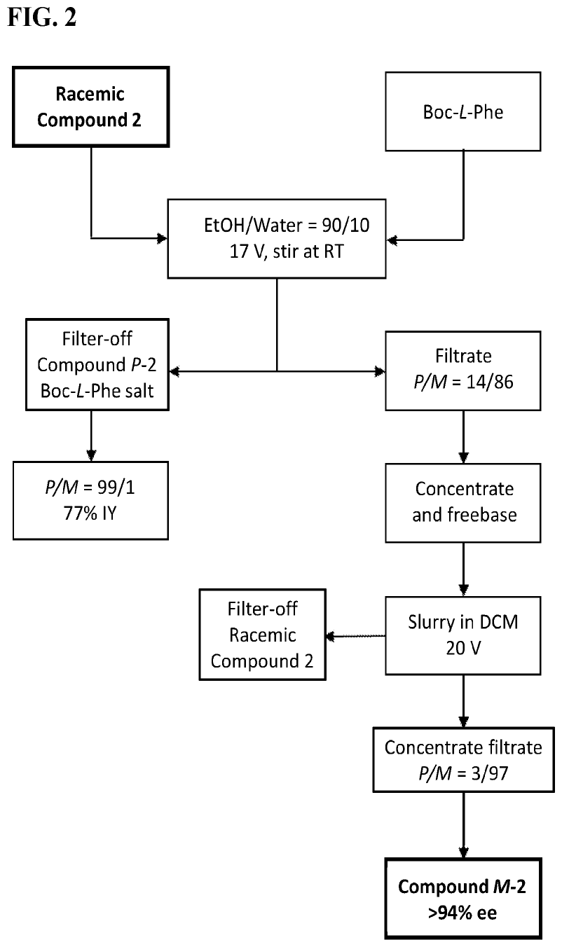

More specifically, N-Boc-L-phenylalanine with THE as a solvent produced a significant increase of the undesired P-enantiomer of Compound 2 (P/M=79/21, 58% diastereomeric excess (de)) compared to other chiral acids. A similar salt formation/crystallization experiment with N-Boc-D-phenylalanine provided enrichment in the MJ-enantiomer of Compound 2 (P/M=7/93, 8600 de). This discovery led to development of two complementary approaches to isolation of the M-enantiomer of Compound 2 from the racemate: using N-Boc-D-phenylalanine via enrichment of solids (FIG. 1); and using N-Boc-L-phenylalanine via enrichment in supernatants (FIG. 2).

In certain aspects of the invention, the undesired P-enantiomer separated in the resolution process can be recycled to provide additional enantiomerically enriched M-enantiomer of Compound 2. The process outlined in the below scheme describes such an approach using the P-enantiomer of Compound 2 Boc-D-phenylalanine salt as the input, however Compound P-2 Boc-L-phenylalanine salt would be suitable as well. Without being bound by a theory, it is believed that this process takes advantage of thermal configurational instability of atropisomers undergoing accelerated racemization at elevated temperatures. Thus, in certain embodiments, a mixture partially enriched in P-enantiomer of Compound 2 (e.g. P/M=85/15) can be racemized (e.g. P/M=49/51) to provide additional M-enantiomer for the resolution step. The procedure directly epimerizes P-enantiomer Compound 2 Boc-D-phenylalanine salt to allow for convenient enantiomer separation after cooling.

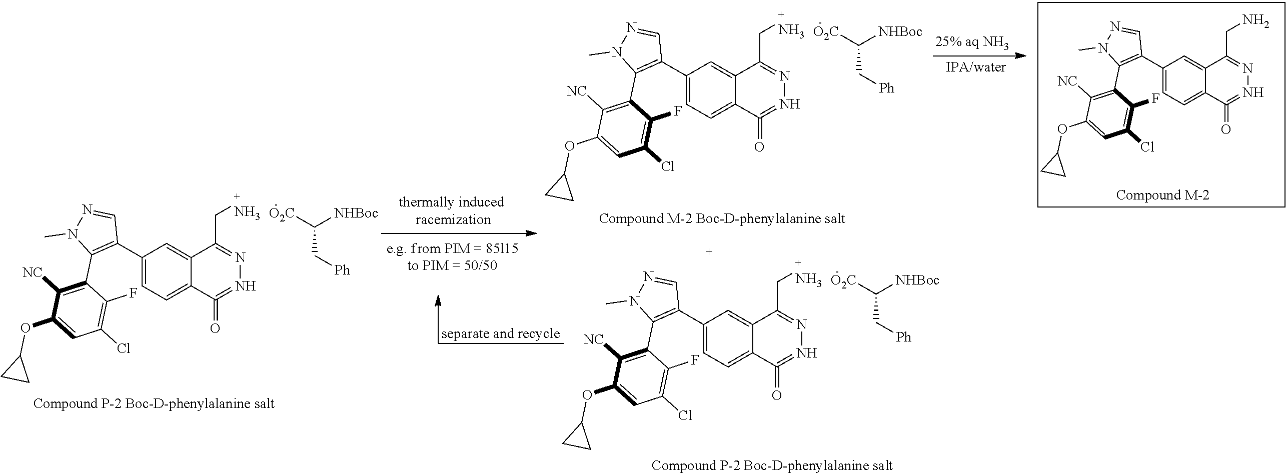

This procedure can be used to continuously recycle the undesired enantiomer to produce additional desired M-2 enantiomer. Thus, for example, recycling of the undesired P-2 enantiomer can include
(a) forming a mixture of a solvent, e.g., aqueous ethanol, M-2, P-2, and Boc-D-phenylalanine in a crystallization vessel to form Compound M-2 Boc-D-phenylalanine salt and Compound P-2 Boc-D-phenylalanine salt;
(b) forming a solid phase and a liquid phase in the mixture, where the solid phase contains an enantiomeric excess of Compound M-2 Boc-D-phenylalanine salt and the liquid phase contains an enantiomeric excess of Compound P-2 Boc-D-phenylalanine salt;
(c) filtering a portion of the liquid phase;
(d) subjecting the filtered liquid phase portion to conditions sufficient to produce an increased amount of the Compound M-2 Boc-D-phenylalanine salt in the filtered liquid phase relative to the amount of that salt in the liquid phase in (b); (e) returning the filtered liquid phase produced in (d) to the crystallization vessel; and
(f) isolating crystals of Compound M-2 Boc-D-phenylalanine salt.

In this procedure, if the solid phase does not form in (b) without cooling, the process can include adjusting the temperature of the mixture from (a) to a temperature in which the Compound M-2 Boc-D-phenylalanine salt and the Compound P-2 Boc-D-phenylalanine salt have different solubilities.

It was also surprisingly discovered that the N-Boc-L-phenylalanine and N-Boc-D-phenylalanine salts of the M- and P-enantiomers of Compound 1 demonstrated differential solubilities.

In addition to separating atropisomers of Compound 2, the methods disclosed herein can readily be used to separate atropisomers of other compounds, including biologically active compounds and intermediates useful for preparing such biologically active compounds. Specific examples of compounds that exist in atropoisomeric form and to which the methods disclosed herein can be applied include [1,1′-binaphthalene]-2,2′-diol and 6-fluoro-7-(2-fluoro-6-hydroxyphenyl)-(1M)-1-[4-methyl-2-(propan-2-yl)pyridin-3-yl]-4-[(2S)-2-methyl-4-(prop-2-enoyl)piperazin-1-yl]pyrido[2,3-d]pyrimidin-2(1H)-one and synthetic intermediates useful for manufacturing this compound.

In certain aspects, this invention provides methods of separating a mixture of atropisomers (Embodiment A), wherein the methods comprise:
(a) forming a mixture of a solvent, a first atropisomer and a second atropisomer in a crystallization vessel;
(b) producing a liquid phase and a solid phase by, if necessary, adjusting the temperature of the mixture to a temperature in which the first atropisomer and second atropisomer have different solubilities, where the solid phase contains an enantiomeric excess of the first atropisomer and the liquid phase contains an enantiomeric excess of the second atropisomer;
(c) removing a portion of the liquid phase;
(d) subjecting the removed liquid phase portion to conditions sufficient to produce an increased amount of the first atropisomer in the removed liquid phase relative to the amount in the liquid phase in (b);
(e) returning the removed liquid phase produced in (d) to the crystallization vessel; and
(f) isolating crystals of the first atropisomer.

In certain aspects, depending on the temperature of the vessel, solvent, and starting materials and the choice of solvent, the atropisomer with lower solubility will crystallize without cooling or seeding. Where necessary, cooling and/or seeding may be employed to produce the solid and liquid phases.

In certain aspects of Embodiment A, the atropisomers are of a compound having the Formula I (Embodiment B)

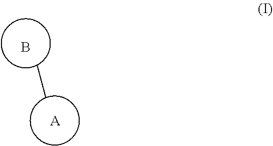

wherein
the A-ring is

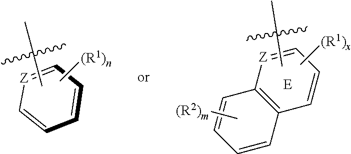

wherein the point of attachment of the bicyclic A-ring to the B-ring is on ring E;
the B-ring is a 6-10 membered aryl group, 5-10 membered heteroaryl group, 5-10 membered heterocycloalkyl group, an amide group, a sulfone group, a sulfoxide group, an olefin group, an amine group, or an ether group, each of which is optionally substituted;
Z is N, CH or CR¹;
n is 0, 1, 2, 3, or 4;
x is 0, 1, or 2;
m is 0, 1, 2, 3 or 4;
   each R¹ is independently C₁-C₆ alkyl, C₁-C₆ alkoxy, C₃-C₈ cycloalkyl, C₃-C₈ cycloalkyloxy, halogen, cyano, hydroxy, amino, or mono- or di-C₁-C₆ alkyl amino; and
   each R² is independently C₁-C₆ alkyl, C₁-C₆ alkoxy, C₃-C₈ cycloalkyl, C₃-C₈ cycloalkyloxy, halogen, cyano, hydroxy, amino, or mono- or di-C₁-C₆ alkyl amino;
   provided that at least one of the positions on the A-ring ortho to the point of attachment of the A-ring to the B-ring is substituted with R¹.

In a particular embodiment of Embodiments A and B (Embodiment C), the removing, subjecting and returning are continued until the amount of the second atropisomer in the liquid phase is below a pre-determined level (Embodiment C).

In a particular embodiment of Embodiments A-C(Embodiment D),
the mixture of the solvent, the first atropisomer and the a second atropisomer further comprises a resolving agent which forms a first atropisomer-resolving agent complex and a second atropisomer-resolving agent complex in the mixture; and
the method further comprises subjecting the crystals of the first atropisomer-resolving agent complex to conditions capable of separating the first atropisomer from the first atropisomer-resolving agent complex.

In a particular embodiment of Embodiments A-D (Embodiment E), the first atropisomer is (2M)-2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile (Compound M-2) and the second atropisomer is (2P)-2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile (Compound P-2).

In a particular embodiment of Embodiments A-E (Embodiment F), the mixture of solvent, first atropisomer and second atropisomer further comprises a resolving agent which is N-Boc-D-phenylalanine.

In a particular embodiment of Embodiments A-F (Embodiment G), the solvent is MeOH or aqueous MeOH.

In a particular embodiment of Embodiments A-F (Embodiment H), the solvent is EtOH or aqueous EtOH,

In a particular embodiment of Embodiments A-F (Embodiment I), the solvent is about 85:15 (v/v) to about 99:1 (v/v) EtOH/water.

In a particular embodiment of Embodiments A-I (Embodiment J), the adjusting in (b) is to a temperature of about 20-25° C.

In a particular embodiment of Embodiments A-J (Embodiment K), the subjecting in (d) comprises heating at a temperature of from about 80-200° C.

Particular B-ring heteroaryl groups in Formula I include triazolyl, pyrazolyl and imidazolyl.

Particular B-ring heterocycloalkyl groups in Formula I include pyrimidinonyl, pyridinonyl, piperazinyl, piperidinyl, morpholyl, pyrrolidinyl, tetrahydronpyranyl, 2-oxopyrido[2,3-d]pyrimidin-1(2H)-yl, 2-oxo-3,4-dihydro-1,8-naphthyridin-1(2H)-yl, and 7-oxo-5,6,7,8-tetrahydroquinolin-8-yl.

Particular B-ring aryl groups in Formula I include phenyl and naphthyl, each of which is optionally substituted with one or more of C₃-C₆ alkyl, hydroxy, cyano, or halogen.

Particular A-ring groups in Formula I include phenyl, pyridyl, naphthyl substituted in at least one ortho position relative to the point of attachment to the B-ring with C₃-C₆ alkyl, hydroxy, cyano, or halogen.

Common abbreviations used herein include:
e.e.—enantiomeric excess
d.e.—diastereoisomeric excess
e.r.—enantiomeric ratio
d.r.—diastereoisomeric ratio
SFC—Supercritical Fluid Chromatography
SPPS—Solid Phase Peptide Synthesis
Boc—tert-butyloxycarbonyl
DCM—dichloromethane
EtOAc—ethyl acetate
EtOH—ethanol
IPA, i-PrOH—propan-1-ol, iso-propyl alcohol
MeCN—acetonitrile
MeOH—methanol
Phe—phenylalanine
D-Phe—D-phenylalanine
L-Phe—L-phenylalanine
THE—tetrahydrofuran

Thus in one embodiment of the invention (Embodiment L), there is provided a method of separating a mixture of M and P enantiomers of Compound 2, the method comprising the steps of. (a) contacting the mixture with N-Boc-D-phenylalanine to form a mixture of M-enantiomer N-Boc-D-phenylalanine salt and P-enantiomer N-Boc-D-phenylalanine salt; (b) filtering the mixture to obtain a solid phase enriched in the M-enantiomer N-Boc-D-phenylalanine salt; and (c) reacting the solid phase with excess NH₃, or other base, to obtain a solid enriched in M-enantiomer free base Compound 2.

Alcohol solvents include, but are not limited to, alcohols having from 1 to 6 carbon atoms, including methanol (MeOH), ethanol (EtOH), n-propanol, isopropanol, n-butanol, 2-butanol, t-butanol, n-pentanol, 2-pentanol, 3-pentanol, n-hexanol, etc. In certain such embodiments, the contacting in step (a) occurs in a MeOH or aqueous MeOH. Suitable aqueous methanol mixtures have up to about 25% by volume of water.

In certain such embodiments, the contacting in step (a) occurs in a EtOH or aqueous EtOH. Suitable aqueous ethanol mixtures for batch processing have up to about 30% by volume of water. Preferred aqueous ethanol mixtures for batch processing are about 90:10 (v/v) ethanol/water. For continuous or semi-continuous processing, ethanol having from 0-15% (v/v/) water is preferred, 0-10% water (v/v) is more preferred, and 0-5% water is particularly preferred.

In certain embodiments, the contacting in step (a) occurs in pure THE or THF/alcohol, for instance THF/EtOH at a volume ratio of 10:1, 9:1, 8:1, 7:1, 6:1, 5:1, 4:1 or 3:1.

In certain such embodiments, the solid phase obtained in step (b) is washed one or more times with additional solvent to remove P-enantiomer N-Boc-D-phenylalanine salt.

In certain such embodiments, the reacting step (c) occurs in an iso-propyl alcohol (IPA, i-PrOH) solution, or isopropyl alcohol and water solution. Water-rich mixtures of IPA-water (e.g. 90/10 v/v) are preferred to maximize recovery and control API form. The desired API form (Free Base Type A) is the most stable at water activity α_{w}≥0.13. In certain embodiments, the step (c) solution is optionally seeded with M-enantiomer free base Compound 2.

In certain embodiments, the enriched solid obtained in step (c) is obtained by filtering the reaction mixture after M-enantiomer enriched free base Compound 2 precipitates.

In certain embodiments, the filtered M-enantiomer enriched free base Compound 2 precipitate is purified by additional slurry or washing steps designed to remove residual N-Boc-D-phenylalanine.

In a particular embodiment of Embodiment L (Embodiment M) the contacting generates a slurry comprising
a solid phase enriched with the Compound M-2 N-Boc-D-phenylalanine salt, and
a liquid phase enriched with the Compound P-2 N-Boc-D-phenylalanine salt; and
   the method further comprises
separating a portion of the liquid phase from the slurry, and
heating the liquid phase to produce a heated mixture having a ratio of Compound P-2 N-Boc-D-phenylalanine salt to Compound M-2 N-Boc-D-phenylalanine salt that is different than the ratio prior to the heating.

In a particular embodiment of Embodiment M (Embodiment N) the ratio after the heating is approximately that of a racemic mixture of the Compound M-2 and Compound P-2 N-Boc-D-phenylalanine salts.

In a particular embodiment of Embodiment M or Embodiment N (Embodiment O), the method further comprises cooling the heated mixture to produce a cooled mixture comprising a solid phase enriched with the Compound M-2 N-Boc-D-phenylalanine salt and a liquid phase enriched with the Compound M-2 N-Boc-D-phenylalanine salt.

In a particular embodiment of Embodiment O (Embodiment P), the cooling of the heated mixture comprises combining the heated mixture with the slurry.

In certain embodiments, filtrate from step (b) enriched with P-Compound 2 N-Boc-D-phenylalanine salt is used to obtain a racemic or nearly racemic mixture of P- and M-Compound 2 N-Boc-D-phenylalanine salts. Typically this is done by heating the filtrate to induce racemization. Racemization can be followed by precipitation of the Compound M-2 N-Boc-D-phenylalanine salt under favorable solvent conditions.

In another embodiment of the invention, there is provided a method of separating a mixture of M- and P-Compound 2 enantiomers, the method comprising the steps of (a) contacting the mixture with N-Boc-L-phenylalanine to form a mixture of M-enantiomer N-Boc-L-phenylalanine salt and P-enantiomer N-Boc-L-phenylalanine salt; (b) filtering the solid phase to obtain a liquid phase enriched with the M-enantiomer N-Boc-L-phenylalanine salt; and (c) reacting the M-enantiomer N-Boc-L-phenylalanine salt with base to obtain a M-enantiomer enriched free base Compound 2.

In certain such embodiments, the contacting in step (a) occurs in a EtOH or EtOH/water solvent.

In certain such embodiments, the contacting in step (a) occurs in a MeOH or MeOH/water solvent.

In certain such embodiments, the contacting in step (a) occurs in a THE or THF/water solvent.

In certain such embodiments, the aqueous phase obtained in step (b) is concentrated to dryness to obtain solid M-enantiomer enriched N-Boc-L-phenylalanine salt.

In certain such embodiments, the reacting step (c) occurs in a suspension of water and dichloromethane.

In certain such embodiments, the reacting step (c) occurs in other than a suspension of water and dichloromethane.

In certain embodiments, the enriched M-enantiomer free base Compound 2 obtained in step (c) is slurried, for instance with dichloromethane and filtered to remove racemic free base Compound 2, to obtain a further enriched M-enantiomer free base Compound 2 in solution.

In certain embodiments, the further enriched M-enantiomer free base Compound 2 solution is concentrated to obtain solid further enriched M-enantiomer free base Compound 2.

In another embodiment, the invention provides a system for separating atropisomers comprising a crystallization module, an epimerization module and a collection module.

In particular embodiments of the system, the crystallization module is fluidly connected to the epimerization module by a removal channel and a return channel.

In certain embodiments of the system, the removal channel comprises the collection module, the collection module is fluidly and directly connected to the epimerization module and the crystallization module, and the return channel is fluidly and directly connected to the crystallization module and the epimerization module.

In other particular embodiments of the system, material is continuously or semi-continuously removed from the crystallization module and fed directly or indirectly into the epimerization module, and material is at least semi-continuously returned from the epimerization module to the crystallization module.

The invention further provides a method for separating atropisomers comprising
selectively crystallizing a less soluble atropisomer in a crystallization module; and
epimerizing a more soluble atropisomer in an epimerization module;
wherein soluble material is continuously or semi-continuously provided from the crystallization module directly or indirectly to the epimerization module, and material from the epimerization module is at least semi-continuously returned to the crystallization module.

In particular embodiments, the selectively crystallizing comprises introducing into the crystallization module a solvent and a mixture of first and second atropisomers, where the first atropisomer and second atropisomer have different solubilities in the solvent, and optionally adjusting the temperature to cause crystallization of the less soluble atropisomer.

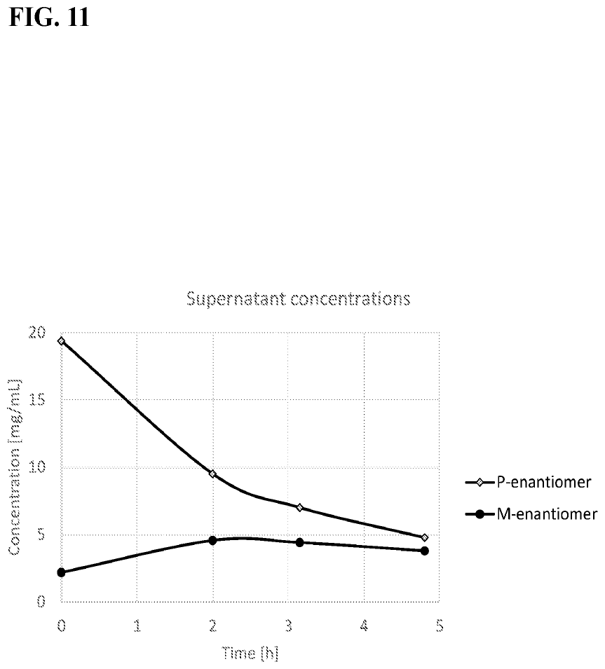

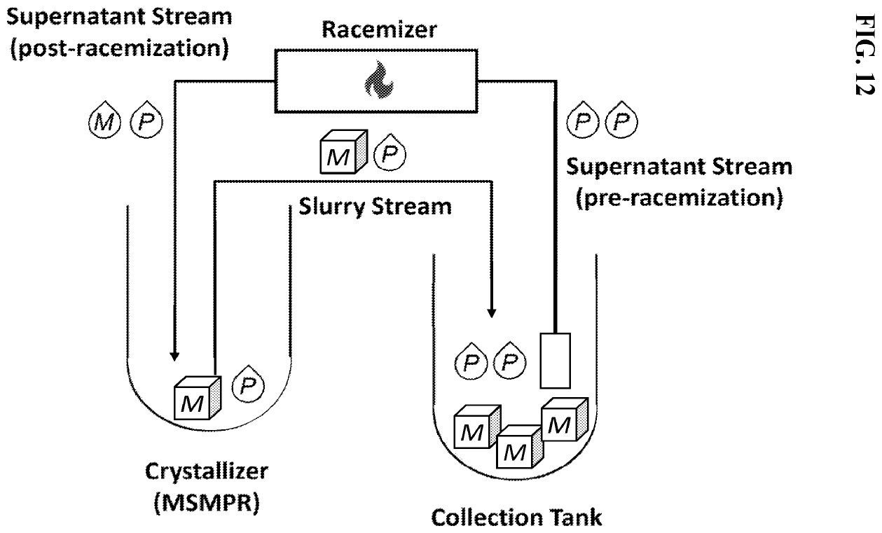

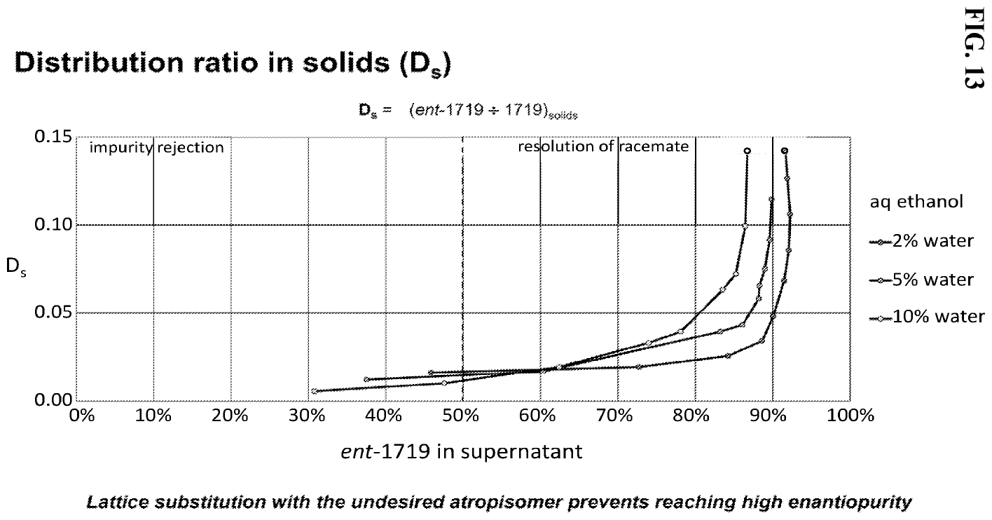

In particular embodiments, the system, includes a crystallization module (Crystallizer) between the epimerization module (racemizer) and the collection module or tank. See FIG. 12. This arrangement can significantly reduce the time necessary to reach the equilibrium. See FIG. 11.). Feeding the racemized stream from the epimerization module (racemizer) directly into the crystallization module (MSMPR vessel (Mixed Slurry Mixed Product Reactor)) allows the resolution process to be operated in the most desirable regime within the crystallizer. See FIG. 13. Without being bound by particular theory, it is believed that the kinetics of crystallization are improved because the process is operated at the highest achievable supersaturation of the desired diastereoisomer. In addition, since the crystallization occurs from a closely racemic supernatant, lattice substitution with the undesired diastereoisomer is minimized leading to highly diastereomerically pure crystals (FIG. 13).

In particular embodiments, the epimerizing comprises subjecting the more soluble atropisomer to conditions sufficient to produce an increased amount of the less soluble atropisomer.

### EXAMPLES

The following Examples are intended to illustrate further certain embodiments of the invention and are not intended to limit the scope of the invention.

### Example 1A: Process for Increasing the Proportion of Solid-Phase M-Enantiomer of Compound 2 Using Boc-D-Phenylalanine

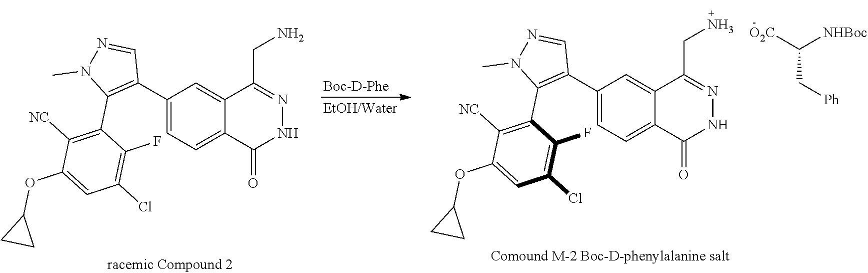

A mixture of EtOH (700 mL) and water (80 mL) was prepared in a four-necked round-bottomed flask at 23° C. Racemic Compound 2 (65.0 g, 140 mmol, 1.00 equiv) and Boc-D-Phe-OH (39.4 g, 153.8 mmol, 1.1 equiv) were charged into the flask and the mixture was stirred at ambient temperature. After approximately 15 min., a large portion of the solids dissolved to result in a light-yellow suspension. The stirring was continued at 20-25° C. for 16 h during which the reaction mixture gradually turned into a thick white slurry. A sample of the suspension was pulled and filtered (filter-cake: 84.0% e.e.; filtrate: −70.0% e.e.). The reaction mixture was filtered at 20-25° C. and the filter-cake was washed with EtOH (130 mL) to afford 66.5 g of a white wet solid (filter-cake: 92.8% e.e.; filtrate: −75.7% e.e.). The wet-cake was resuspended in a mixture of EtOH (300 mL) and water (55 mL) at 23° C. and stirred at 20-25° C. for 18 h. A sample of the suspension was pulled and filtered (filter-cake: 97.1% e.e.; filtrate: 60.7% e.e.). The slurry was filtered at 20-25° C., the filter-cake was washed with EtOH (60 mL) to afford 46.5 g of white wet solid (97.5% e.e., 84.1% w/w by Q-NMR, 38.3% assay yield).

### Example 1B: Process for Freebasing of M-Enantiomer of Compound 2 Boc-D-Phenylalanine Salt to Obtain the Free Base Compound M-2 (Free Base Type A)

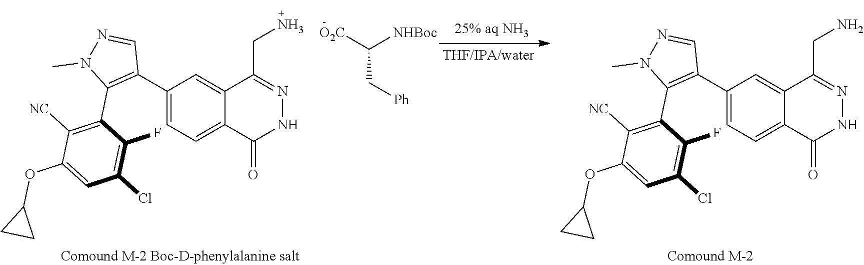

A mixture of THE (45 mL) and water (15 mL) was prepared in a four-necked round-bottomed flask at 23° C. Compound 2 Boc-D-Phe salt (10 g, 97.5% e.e., 84.1% w/w by Q-NMR) was added and allowed to dissolve to afford a clear light-yellow solution. Another 1 L four-necked round-bottomed flask was charged with H₂O (390 mL) and aq. NH₃ (25% w/w, 10 mL). The solution of the salt in aq THE was added dropwise to the 1 L four-necked round-bottomed flask. Gradual formation of white solid occurred during the addition. The resulting suspension was stirred at 20-25° C. for 17 h. The slurry was filtered at 20-25° C. and the filter-cake was washed with water (2×40 mL) to afford 9.36 g of white wet-cake. The wet-cake was re-slurried in a mixture of IPA/water (10/90 v/v, 80 mL) at 20-25° C. for 3 h and filtered. The filter-cake was dried at 45° C. for 12 h to afford 5.70 g of white solid (99.9% HPLC purity, 97.5% e.e., 5.80% water by KF, 91.7% w/w by Q-NMR, Free Base Type A by XRD, 91.7%, 97.8% isolated yield).

### Example 2: Process for Increasing the Proportion of M-Enantiomer of Compound 2 in the Supernatant Using Boc-L-Phenylalanine

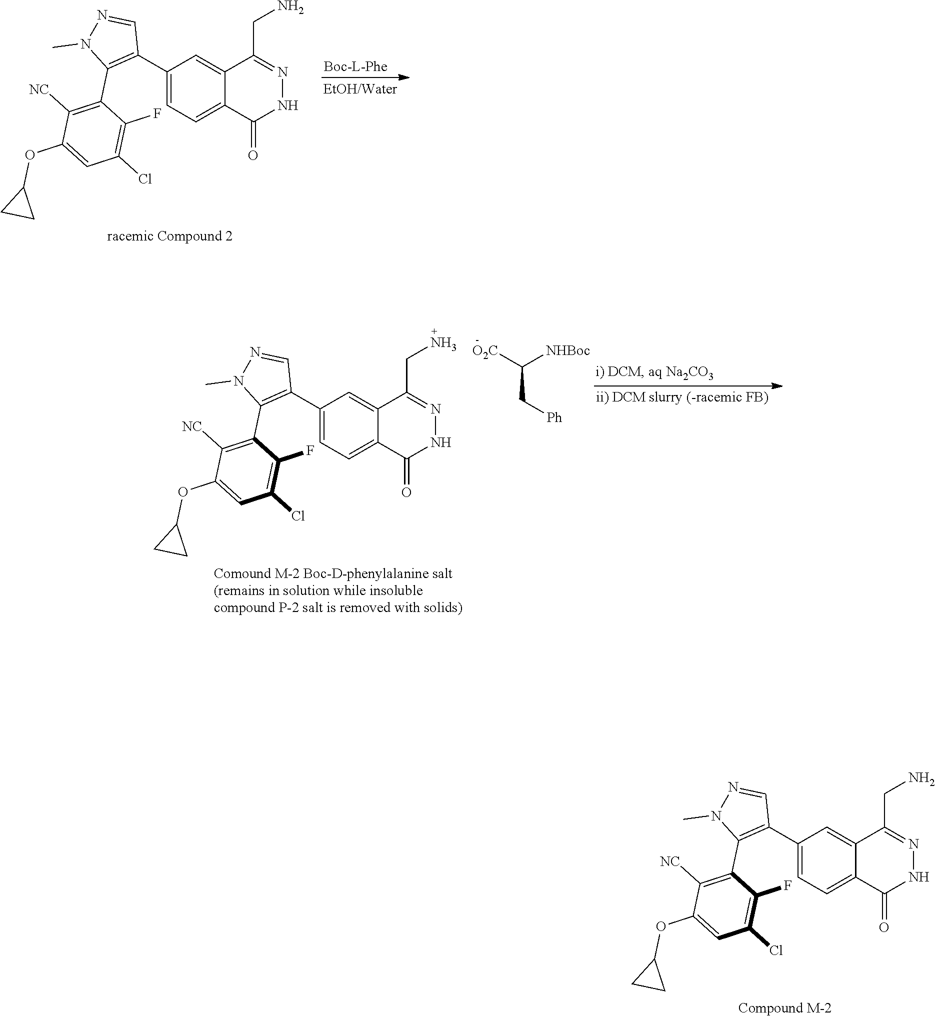

Compound 2 racemic freebase (1.8 g, 3.9 mmol) and Boc-L-Phe (1.03 g, 3.9 mmol) were placed into a 100 mL glass vessel. To this mixture, EtOH/water (90/10 v/v, 30 mL) was added at 25° C. The resulting slurry was agitated using an overhead stirrer at 350 rpm at 25° C. During the initial 15 minutes, the solid material dissolved resulting in a hazy solution. The reaction mixture was continued to be stirred at 20° C. for 24 h during which a thick milky slurry was obtained. The reaction mixture was filtered. The undesired enantiomer (˜75% of total) was rejected in the solids as Compound P-2 Boc-L-Phe salt (−92% e.e.) and the desired enantiomer was enriched in the filtrate as Compound M-2 Boc-L-Phe salt (68% e.e.). The filtrate was rotavapped to dryness under reduced pressure at 35° C. to afford the crude salt as a yellowish solid. The resulting crude salt was freebased using a mixture of water (30 mL) and DCM (30 mL) with sufficient of saturated Na₂CO₃ to obtain a pH between 8-10. The mixture was allowed to stir at 25° C. for 4 h. The organic layer was separated using separating funnel and rotavapped to dryness under reduced pressure at 35° C. to afford partially upgraded Compound M-2 as a yellowish poorly crystalline solid (1.00 g, 68% e.e., 97% LC purity). The enantiomeric purity of the freebase was further upgraded by slurry conditioning in DCM. The solids were re-suspended in DCM (20 mL) and stirred at 25° C. for ˜72 h. The remaining solids were filtered-off and confirmed to be a nearly racemic Compound 2 (10% e.e.). The concentrated filtrates afforded upgraded Compound M-2 (0.72 g, 94% e.e., 39% yield).

### Example 3: Process for Recycling P-Enantiomer of Compound 2 Boc-D-Phenylalanine Salt Streams

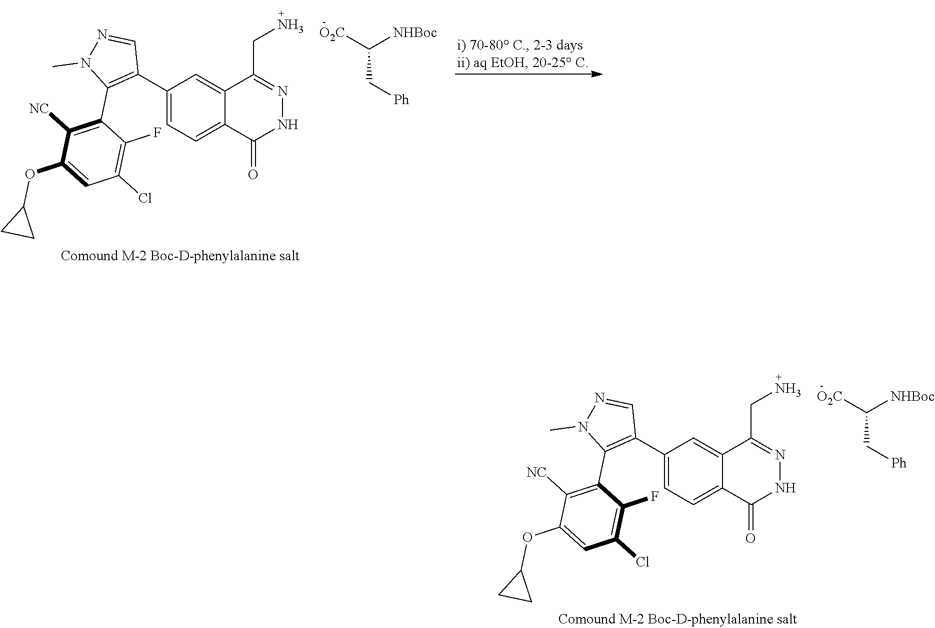

The first aq EtOH filtrate from the resolution process described in Example 1A (100 g, 3.9 g freebase assay, −650.4% e.e.) was charged to a four-necked round-bottomed flask. The solution heated at 70-80° C. for 60 h. to racemize the salt. The resulting nearly racemic mixture (-6.0% e.e.) was combined with the remaining liquid streams from the resolution process described in Example 1A (50 g, 1.3 g freebase assay, 40.6% e.e.; 25 g, 0.5 g freebase assay, 63.0% e.e.) and concentrated to about −30 mL at 30-45° C. under reduced pressure. EtOH (50 mL) was added and the mixture was distilled to −30 mL. This operation was repeated one more time. EtOH (50 mL) was added and the mixture was stirred at 70-75° C. for 0.5 h. The mixture was cooled to room temperature to trigger the crystallization of Compound M-2 Boc-D-Phe salt. The resulting suspension was aged at 20-25° C. until no further desupersaturation was observed by assaying the supernatant. The reaction mixture was filtered and the resulting filter-cake was washed with EtOH (10 mL) and dried to afford 3.13 g of Compound M-2 Boc-D-Phe salt as white solid (99.8% LC purity, 96.0% e.e., 97.4% w/w by Q-NMR, 35% isolated yield).

### Example 4

Advanced intermediates Int_{AB} and Int_{CD} were coupled using palladium catalyzed Suzuki-Miyaura reaction to afford racemic mixture of Boc-protected species. The desired M-enantiomer Boc-protected species was separated from the undesired P-enantiomer using preparative chiral chromatography. The final Compound 2M-enantiomer product was obtained after acid-promoted removal of the Boc protecting group. This process of chiral chromatographic separation is disadvantageous because it is solvent intensive, non-scalable and expensive.

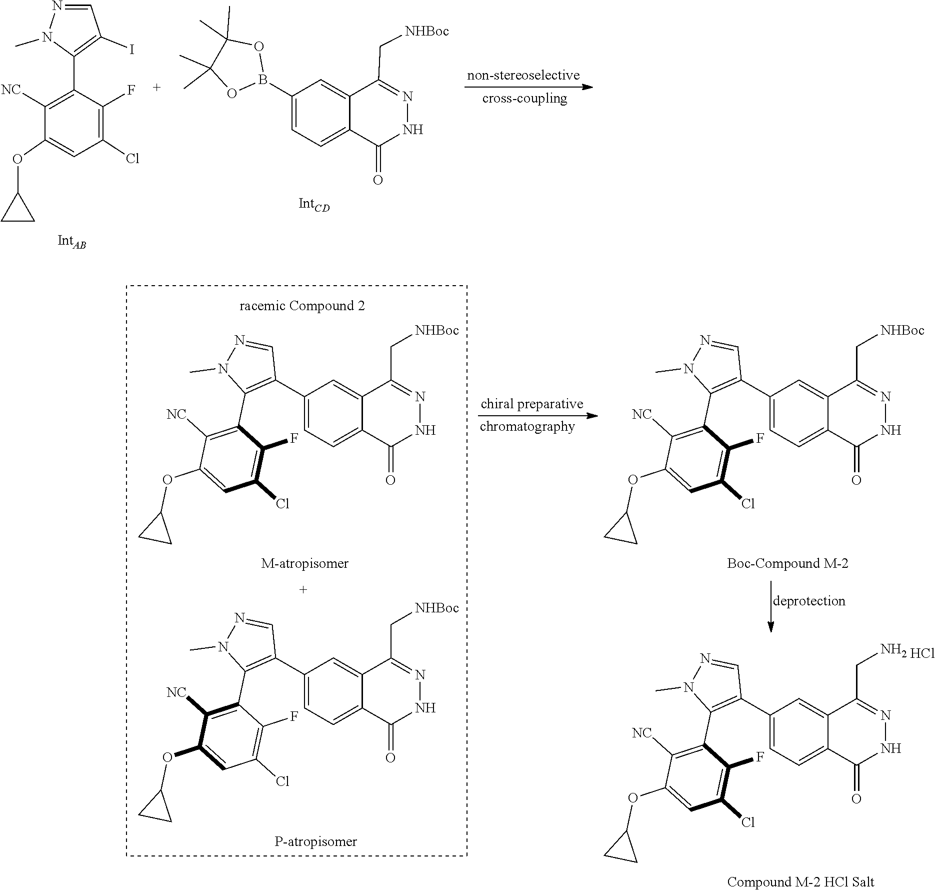

Coupling: To A mixture of Int_{AB} (200 g, 479 mmol, 1.00 equiv), Int_{CD} (231 g, 575 mmol, 1.2 equiv) and aqueous K₃PO₄ (1.5 M, 958 mL, 3 equiv) in dioxane (1.80 L), then Ad₂-n-Pd-G3 (24.4 g, 33.5 mmol, 0.07 equiv) was added under N₂. The mixture was stirred at 80° C. for 16 h under N₂. The mixture was cooled to room temperature, then poured into ice-water (2.00 L) and stirred for 30 min. The aqueous phase was extracted with ethyl acetate (2 x 1.00 L). The combined organic phase was washed with brine (1.00 L), dried with anhydrous Na₂SO4, filtered and concentrated in vacuum. The residue was purified by column chromatography (SiO₂, Petroleum ether/Ethyl acetate=5/1 to 1/1) to give racemic Compound 2 (192 g, 354 mmol, 70.0% yield) as light yellow solid.

Chiral separation by preparative chromatography: Racemic Compound 2 (3.00 kg, 5.31 mol, 1.00 equiv) was separated by SFC (Shimadzu Mobile Phase: 45% EtOH (0.1% NH₃-H₂O) in hexane Flow Rate: 140 g/min, Cycle Time: 9 min, total time: 4500 min, Single injection volume: 16.0 mL) to afford Boc-Compound M-2 (1.37 kg, 2.42 mol, 45.6% yield, 98.3% e.e., 98.2% purity) as an off-white solid.

Final deprotection to produce Compound M-2 HCl Salt: To a mixture of Boc-Compound M-2 (200 g, 354 mmol, 1 equiv) in MeOH (1.00 L) was added HCl/MeOH (4 M, 250 mL, 2.82 equiv) in one portion at 20° C. under N₂. The mixture was stirred at 20-35° C. for 2 h. The reaction mixture was concentrated under reduced pressure to give a residue, then switched the solvent with EtOAc three times. The residue was suspended in EtOAc (1.00 L, 5.00 V) and stirred at 15° C. for 16 h. The deprotection procedure was carried-out in seven (7) replicates in parallel. All the batches were combined, filtered, and the filter-cake was dried under reduced pressure to an off-white solid. The solid was added to deionized water/MeOH (12/1, 10 L) and stirred for 3 hours. The solution was lyophilized to give Compound M-2 HCl Salt (1050 g, 2.09 mol, 99.1% purity, 99.1% e.e., 84.5% yield) as an off-white solid.

¹H NMR (400 MHz, DMSO-d₆): δ=12.9 (s, 1H), 8.67 (s, 3H), 8.42 (s, 1H), 8.13-8.16 (d, J=12 Hz, 1H), 8.04 (d, J=8.0 Hz, 1H), 7.86-7.87 (d, J=4.0 Hz, 1H), 7.47-7.50 (dd, J=4 Hz, 1H), 4.35 (s, 2H), 4.23-4.28 (m, 1H), 3.79 (s, 3H), 0.94-0.77 (m, 4H).

### Example 5

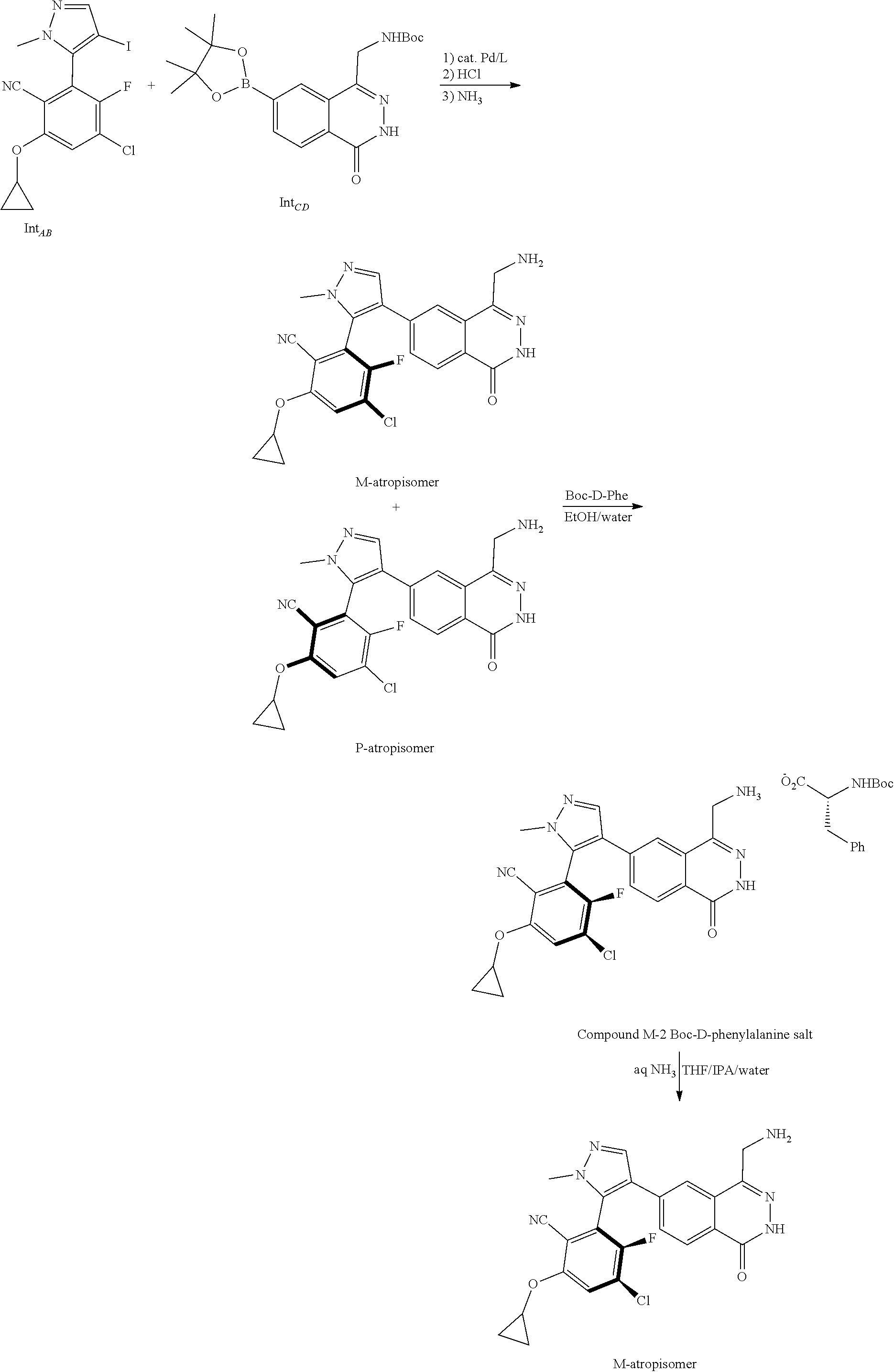

Coupling: Cs₂CO₃ (51.6 g, 158 mmol, 3.30 equiv) and H₂O (80 mL) were combined in a four-necked round-bottomed flask at 20-25° C. and stirred until clear solution was obtained. Int_{AB} (20.86 g, 95.9% w/w, 48.0 mmol, 1.00 equiv), Int_{CD} (23.58 g, 97.8% w/w, 57.5 mmol, 1.20 equiv) and toluene (240 mL) were added. The mixture was vacuum degassed and backfilled with N₂ three times. Ad₂n-Pd-G3 (0.88 g, 2.5 mol %) was added and the mixture was vacuum degassed and backfilled with N₂ three times. The resulting reaction mixture was heated to an internal temperature of 57° C. under N₂. After 27 h at 57° C. analysis of in-process sample indicated 2.7% of Int_{AB} remaining and 87.4% of racemic Boc-Compound 2 formed. The reaction was cooled to 45-50° C. Cysteine (4.0 g, 33 mmol, 0.69 equiv) and 2-MeTHF (40 mL) were added to the mixture and stirring was continued for 6 h at 45-50° C. The reaction mixture was filtered through a short plug of Celite, and the filter cake was washed with 2-MeTHF (100 mL). The aqueous phase was separated. The combined organic phase was washed with 17% aq NaCl (2×100 mL). Anh. MgSO₄ (20 g) and activated charcoal (4.0 g) were added organic solution and the resulting suspension was stirred for 4 h at 50° C. The solids were filtered-off and the waste-cake was washed with 2-MeTHF (100 mL). The combined filtrate was concentrated under reduced pressure at 30-60° C. to ˜40 mL volume. MeOH (60 mL) was added, and the distillation was continued until ˜40 mL volume was reached. This operation was repeated two more times to remove residual toluene and 2-MeTHF. MeOH (140 mL) was added and the mixture was stirred at 50-55° C. for 6 h. The mixture was cooled to 20-25° C. and stirred for another 12 h. The resulting slurry was filtered, and the wetcake was washed with MeOH (40 mL). The obtained wetcake was dried at 60-65° C. for 16 h to afford racemic Boc-Compound 2 as a light-yellow solid (21.84 g, 80.7% yield, 99.7% LC purity).

Boc-deprotection and freebasing of racemate: Racemic Boc-Compound 2 (400 g, 708 mmol) and EtOAc (6.00 L) were charged to a 10 L four-necked round-bottomed flask at 23° C. and stirred for 15 min to allow the material to dissolve affording a light-yellow clear solution. 4 M HCl in EtOAc (2.0 L, 11 equiv) was added dropwise to the stirred mixture at 20-25° C. while colorless solid gradually precipitated out. The resulting suspension was stirred for 21 h at 20-25° C. upon which in-process sample indicated complete Boc-deprotection (0.04% Boc-Compound 2 remaining). The slurry was filtered, and the wetcake was washed with EtOAc (2×0.80 L). The filtercake was dried under reduced pressure at 50° C. for 2 h to afford racemic Compound 2 HCl salt as a colorless solid (398 g, quant. yield, 99.2% LC purity). Racemic Compound 2 HCl salt (398 g) and MeOH (4.00 L) were charged to a 10 L four-necked round-bottomed flask at 23° C. and stirred for 15 min to afford a white suspension. The mixture was cooled to 5-15° C. under N₂. A solution of 7M NH₃ in MeOH (0.400 L) was added dropwise to the mixture at 5-15° C. under N₂. After the addition, the mixture was allowed to warm to room temperature and stirred for another 13 h. The mixture was cooled to 10-15° C. under N₂ and stirred for 1 h to obtain a colorless slurry. The solids were collected by filtration and dried at 50° C. for 18 h to afford racemic Compound 2 freebase as a colorless solid (312.2 g, 96.0% w/w, 99.7% LC purity, 94.9% yield).

Separation of atropisomers: The racemic Compound 2 freebase is then treated as described above in Examples 1A and 1B to obtain the desired Compound M-2.

### Example 6

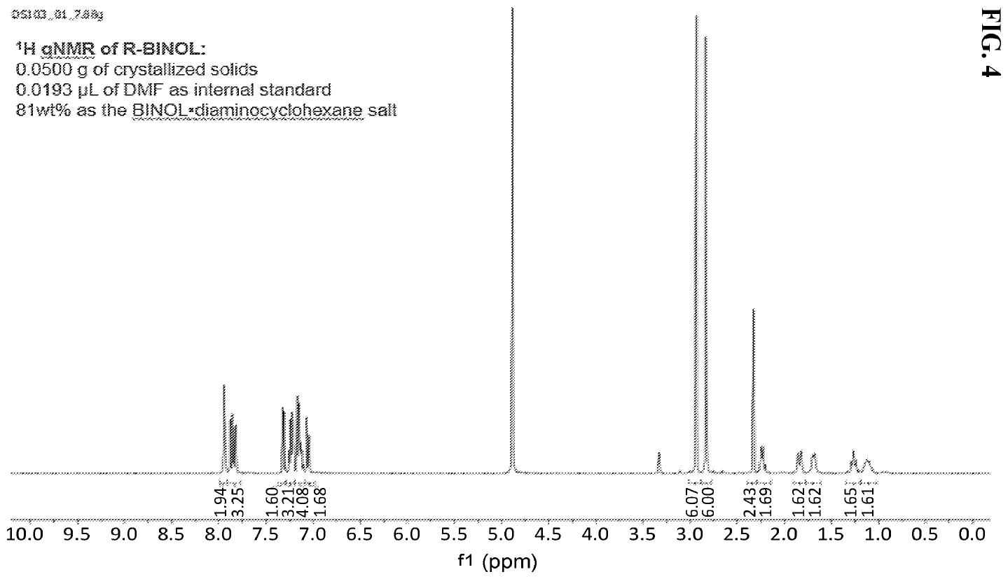

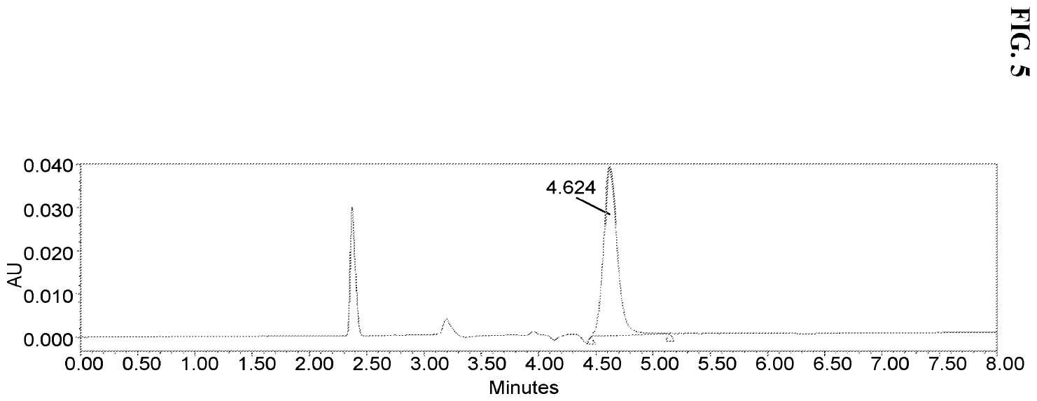

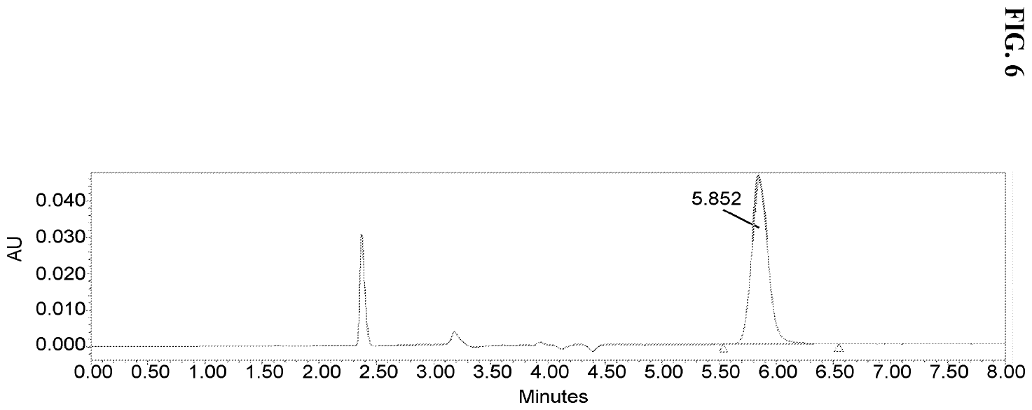

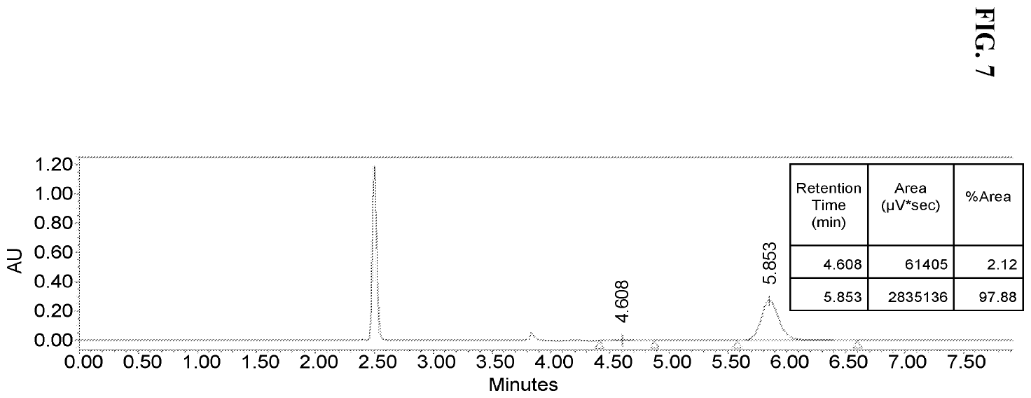

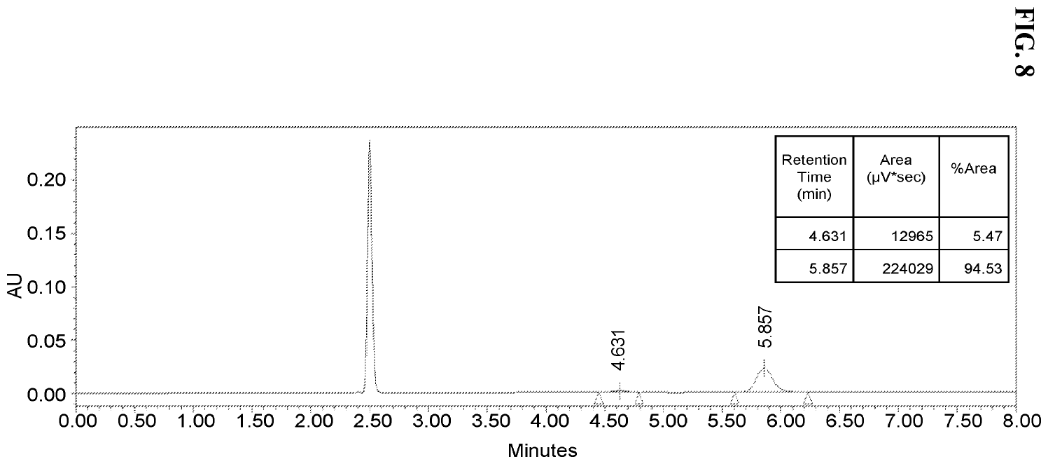

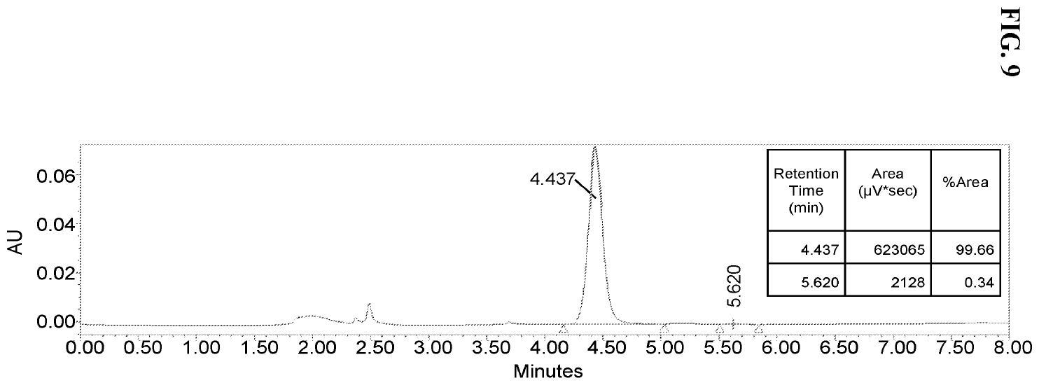

1,1′-Bi-2-napthol (5.71 g, 19.9 mmol, 1 equiv.) was added to an Erlenmeyer flask equipped with a magnetic stir-bar. 80 mL of toluene was added to the flask. The flask was then heated to 80° C. on a hot-plate, while stirring. (1R,2R)-diaminocyclohexane (2.28 g, 19.9 mmol, 1 equiv.) was added to the hot BINOL, and the slurry quickly transitioned to a homogeneous phase. After becoming homogeneous, crystals began to form, heating was turned off, and the mixture was allowed to cool to room temperature and age for 1 hr. A sample of the supernatant was collected for HPLC analysis. The sample contained XYZ of R-BINOL and XYZ of S-BINOL. A 20 μm stainless steel HPLC filter from IDEX was immersed in the toluene. It was connected to an Eldex Optos Model 3 metering pump by 1/8″ O.D. PFA tubing (1/16″ ID.). The pump was connected to a stainless steel plug flow reactor (3.5 mL, 1/8″ O.D, 0.09″ I.D.) by PFA tubing (1/8″ O.D., 1/16″ ID.). Swagelok 1/8″ compression fittings were used to securely connect the tubing. The outlet of the plug flow reactor (PFR) was connected to a 250 psi spring-loaded back pressure regulator from IDEX. The stream flowed out of the bpr and back into the Erlenmeyer crystallization flask. The plug-flow reactor was immersed in mineral oil heated to 200° C., and prior to pumping the BINOL solution through the stainless steel filter and PFR, the recycle loop was pre-filled with pure toluene to facilitate start-up of the recycle procedure. The pump driving the recycle loop was operated at a rate of 3 mL/min, and the recycle was run continuously for 30 hr. After this time, the solids in the flask were filtered. The solids were washed with 5 mL of toluene and left on the filter until dry. 7.88 g of white solid was obtained. The complex was a 1:1:1 mixture of BINOL, diamine and toluene (MW 492 g/mol). Sample was submitted for 1H qNMR analysis to attain the assay weight percent (FIG. 4). Spectrum matched that reported in the literature. It was 99.6% of the 81.0% assay wt. percent of the BINOL/diaminocyclohexane complex corresponding to 99.6 assay wt % of the 1:1:1 complex of BINOL, diamine, and toluene. The assay adjusted isolated yield is 80%. Sample was taken for chiral HPLC analysis. The sample showed 99.65% of R-BINOL and 0.35% of S-BINOL (99.3% ee) (FIGS. 5-9).

### HPLC Conditions for BINOL. Spectra of R and S Isomers of BINOL

| | |
|---|---|
| Column | Chiralpak IC, 5 μm, 4.6 × 250 mm |
| | (Daicel Chiral Technologies, Part No. 83325) |
| Wavelength | 265 nm |
| Column Temperature | 40° C. |
| Column Flow | 1.3 mL/min |
| Acquisition Time | 8 min |
| Isocratic Elution | Hexane:2-Propanol 90:10 (v/v) |
| Injection Volume | 5 μL |
| Retention Times | ~4.5 min (R-BINOL), ~6.0 min (S-BINOL) |

### Example 7: Batch Resolution

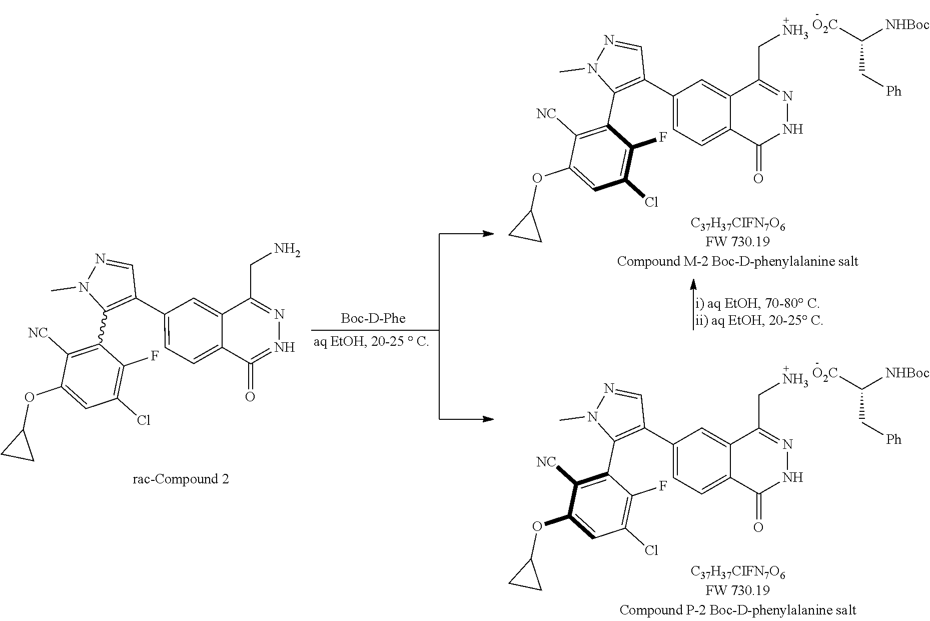

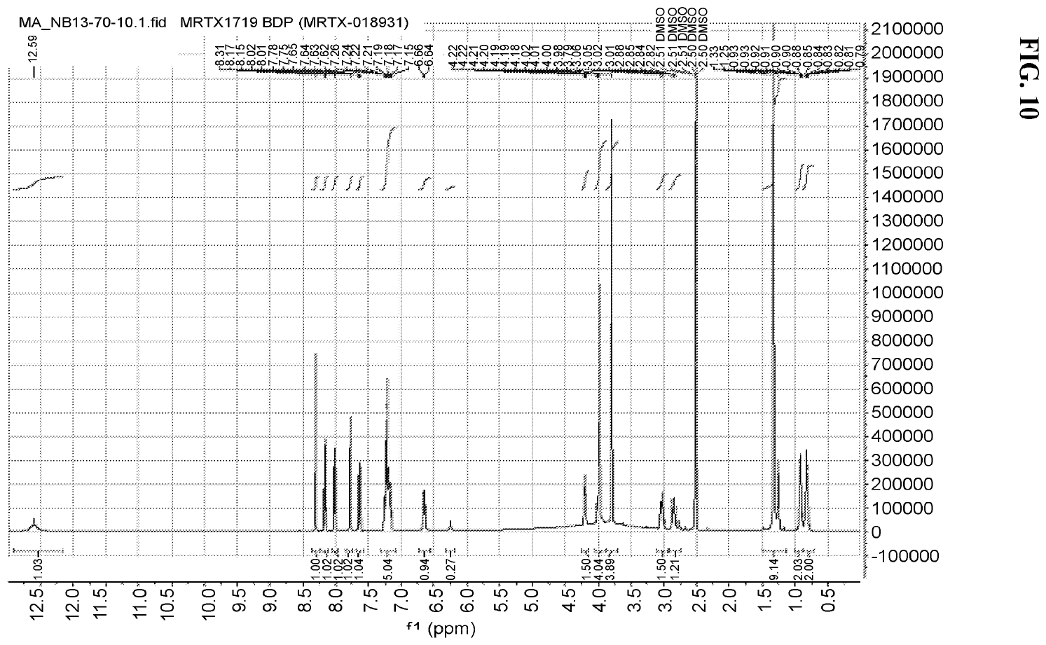

rac-Compound 2 (1.0 equiv) and Boc-D-Phe (1.1 equiv) were allowed to dissolve in EtOH:water (90:10 v/v, 12 V) at room temperature. The resulting supersaturated mixture was seeded with 0.5% w/w of Compound M-2 Boc-D-phenylalanine salt and equilibrated at −22° C. until preferential crystallization of Compound M-2 Boc-D-phenylalanine salt was complete while the supernatant became enriched in the undesired enantiomer salt (Compound P-2 Boc-D-phenylalanine salt). The crystalline solids were separated by filtration and additional reslurry in aqueous EtOH (85:15 v/v, 10 V) was performed to effect additional chiral upgrade (target: S95.5% enantiomeric excess [e.e.] by chiral HPLC). The mother liquors consisting of a mixture of Compound P-2 Boc-D-phenylalanine salt and Compound M-2 Boc-D-phenylalanine salt in aqueous EtOH were thermally racemized (target: <20% enantiomeric excess [e.e.] by chiral HPLC) at 70-80° C. for 24-48 h. Additional crop of Compound M-2 Boc-D-phenylalanine salt was obtained by repeating the aging and reslurry as described above (target: ≥95.5% enantiomeric excess [e.e.] by chiral HPLC). Drying of the resulting crystalline solids at −45° C. afforded Compound M-2 Boc-D-phenylalanine as off white solids in combined 60% yield (first crop: 37% yield; second crop: 23% yield) (FIG. 10).

### HPLC Conditions for Compound 2.

| | |
|---|---|
| Column | Chiralpak IC, 5 μm, 4.6 × 250 mm |
| | (Daicel Chiral Technologies, Part No. 83325) |
| Wavelength | 265 nm |
| Column Temperature | 40° C. |
| Column Flow | 1.3 mL/min |
| Acquisition Time | 8 min |
| Isocratic Elution | Ethanol (0.1% Ethanolamine):Heptane 90:10 (v/v) |
| Injection Volume | 10 μL |
| Retention Times | ~9.7 min (M-2), ~7.4 min (P-2) |

### Example 8: Continuous Resolution

Racemic Compound 2 (92.4 wt %, 10.82 g, 21.5 mmol) was suspended in ethanol and water mixture (EtOH:water 98:2 v/v, 100 mL) in 100 mL EasyMax reactor. Solid Boc-D-phenylalanine (6.25 g, 23.6 mmol, 1.1 equiv) was added and the resulting thick slurry was agitated at 600 rpm using overhead stirrer at room temperature. Within 10 min nearly all the solids went into solution affording a thin yellowish suspension. Thus obtained supersaturated solution of Compound M-2 Boc-D-phenylalanine salt was seeded with crystalline Compound M-2 Boc-D-phenylalanine salt (78 mg, 0.0050 equiv) and allowed to equilibrate with efficient agitation at room temperature. After ˜16 h thick slurry of crystals was obtained. Analysis of the supernatant by chiral LC showed 91.5% of Compound P-2 and 8.5% of Compound M-2-(83% e.e.) with Compound P-2 Boc-D-phenylalanine salt at 39.7 mg/mL (equilibrium solubility of −40 mg/mL) and Compound M-2 Boc-D-phenylalanine salt at 3.7 mg/mL (equilibrium solubility of −4 mg/mL). Having confirmed the mixture is close to equilibrium, continuous resolution was initiated. A 20 μm stainless steel HPLC filter from IDEX was immersed in the crystallization vessel (100 mL EasyMax reactor) containing thick slurry of Compound M-2 Boc-D-phenylalanine salt in ethanol water mixture. It was connected to a Syrris Asia Syringe Pump by 1/16″ O.D. PFA tubing (0.03″ I.D.). The pump was connected to a plug flow reactor made from PFA tubing (1.5 mL, 1/16″ O.D, 0.03″ I.D.). The outlet of the plug flow reactor (PFR) was connected to a 250 psi spring-loaded back pressure regulator from IDEX. The stream flowed out of the bpr and back into the crystallization vessel. The plug-flow reactor was immersed in mineral oil heated to 160° C. The pump driving the recycle loop was operated at a rate of 0.75 mL/min, and the recycle was run continuously for 14.5 h. Intermittent HPLC sampling was conducted. After 14.5 h the productive phase of the continuous resolution was completed as indicated by nearly identical supernatant concentration of Compound P-2 Boc-D-phenylalanine salt and Compound M-2 Boc-D-phenylalanine salt (5.4 mg/mL and 4.3 mg/mL, respectively). The slurry from the crystallizer was filtered. The filtercake was thoroughly deliquored and air-dried to afford technical Compound M-2 Boc-D-phenylalanine salt as off-white powder in 87% yield (14.43 g, 94.2 wt %, 90% d.r., 98.0% LC). Additional reslurry in aq EtOH (EtOH:water 95:5 v/v, 10 V) for 6 h with temperature cycling (20-40° C.) upgraded the chiral purity (96.6 d.r) at the cost of 5% product loss to the liquors.

### Example 9: Continuous Resolution (Separate Crystallization and Collection Modules)

The setup for continuous resolution with separate crystallization and collection modules is depicted in FIG. 12.

Boc-d-phenylalanine (1.37 g, 5.16 mmol, 1.20 equiv) was dissolved in EtOH:water 98:2 v/v (20 mL, 10 vol). Racemic Compound 2 (90.0 wt %, 2.22 g, 4.30 mmol assay) was added portionwise to a well-agitated solution of Boc-d-phenylalanine. The following aliquots of racemic Compound 2 were added allowing for dissolution time in-between charges: 222 mg (portion 1, t=0), 245 mg (portion 2, t=14 min), 267 mg (portion 2, t=36 min). The resulting hazy solution was seeded with Compound M-2 Boc-d-Phenylalanine salt crystals (6 mg, t=42 min). Crystal growth was observed over time resulting in a fluid slurry. The remaining racemic Compound 2 was added portionwise: 290 mg (t=82 min), 233 mg (t=101 min), 271 mg (t=179 min), 694 mg (t=131 min). The resulting thick mixture was diluted with EtOH:water 98:2 v/v (20 mL, 10 vol) (t=144 min) to obtain a fluid slurry. The Collection Module (tank) was charged with the slurry. The supernatant from the Collection Module was drawn through the filter (20 μm, sintered metal) into the Epimerization Module (Racemizer) (150-160° C.) allowing for 2 min residence time (0.75 mL/min flow rate, 1.5 mL Racemizer volume). The racemized output was sent into well-agitated Crystallization Module (Crystallizer) where rapid crystallization of Compound M-2 Boc-d-Phenyalanine salt was taking place resulting in a suspension (slurry). The slurry was peristaltically transferred (pump not shown) out from the Crystallizer while maintaining its volume at −10 mL. The average residence time in the Crystallizer was about 13 min (10 mL, 0.75 mL/min). The slurry was transferred back into the Collection Module thus completing the cycle. The pumping process was performed continuously for 5 hours upon which the supernatant concentration of the undesired P-enantiomer virtually matched the concentration of the desired M-atropisomer signaling the process reached equilibrium. The contents of the system were filtered and the resulting filter-cake was air-dried to afford Compound M-2 Boc-d-Phenylalanine salt as an off-white powder (2.25 g).

Placing the crystallization module between the racemizer and the collection module led to significant reduction of time to reach the equilibrium (FIG. 11).

In this example, as shown in FIG. 12, the equipment includes a comparatively small crystallization module or vessel (crystallizer) directly downstream of the epimerization module (racemizer) and upstream of the collection module (collection tank). Feeding the racemized stream from the epimerization module or racemizer, i.e., a superheated loop, directly into the collection module permits operation of the resolution process to proceed in under the most desirable conditions within the crystallization module. See FIG. 13.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A method of separating a mixture of (2M)-2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile (Compound M-2) and (2P)-2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile (Compound P-2) enantiomers 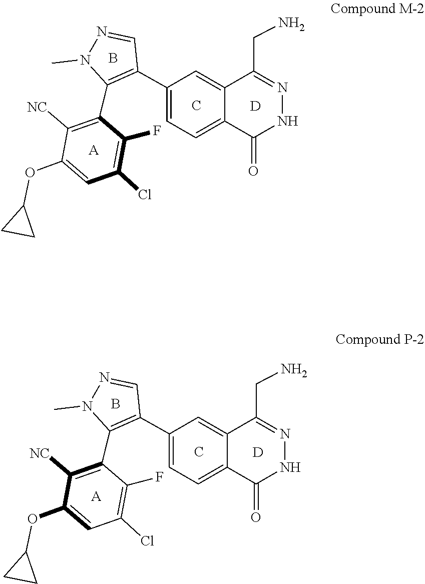
the method comprising the steps of:
(a) contacting the mixture with N-Boc-D-phenylalanine to form a mixture of Compound M-2 N-Boc-D-phenylalanine salt and Compound P-2 N-Boc-D-phenylalanine salt;
(b) filtering the mixture to obtain a solid phase enriched with the Compound M-2 N-Boc-D-phenylalanine salt; and
(c) reacting the solid phase with excess NH₃, or other base, to obtain a solid enriched with M-enantiomer free base of 2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile.

2. The method of claim 1, wherein the contacting in step (a) occurs in MeOH or aqueous MeOH, or in EtOH or aqueous EtOH, or THF or aqueous THF.

3. (canceled)

4. (canceled)

5. (canceled)

6. (canceled)

7. The method of claim 1, wherein the reaction of step (c) occurs in an iso-propyl alcohol solution, or isopropyl alcohol and water solution.

8. (canceled)

9. (canceled)

10. (canceled)

11. The method claim 1, wherein filtrate from step (b) enriched with Compound P-2 N-Boc-D-phenylalanine salt is heated to obtain a racemic or nearly racemic mixture of Compound P-2 and Compound M-2.

12. The method of claim 11, wherein said mixture is separated to obtain pure or enriched Compound M-2 N-Boc-D-phenylalanine salt.

13. A method of separating a mixture of Compound M-2 and Compound P-2 enantiomers, the method comprising the steps of:
(a) contacting the mixture with N-Boc-L-phenylalanine to form a mixture of Compound M-2 N-Boc-L-phenylalanine salt and Compound P-2 N-Boc-L-phenylalanine salt;
(b) filtering the solid phase to obtain a liquid phase enriched with the Compound M-2 N-Boc-L-phenylalanine salt; and
(c) reacting the Compound M-2 N-Boc-L-phenylalanine salt with base to obtain a solid free base Compound M-2.

14. The method of claim 13, wherein the contacting in step (a) occurs in MeOH or aqueous MeOH, or in EtOH or aqueous EtOH, or THF or aqueous THF.

15. (canceled)

16. (canceled)

17. (canceled)

18. (canceled)

19. The method claim 1, wherein step (c) occurs in a suspension of water and dichloromethane or step (c) occurs in other than a suspension of water and dichloromethane.

20. (canceled)

21. (canceled)

22. A method of obtaining substantially pure or enriched Compound M-2 the method comprising the steps of:
(a) obtaining a mixture of Boc-protected Compound M-2 and Boc-protected Compound P-2;
(b) chromatographically separating the Boc-protected Compound M-2 from the Boc-protected Compound P-2; and
(c) deprotecting the Boc-protected Compound M-2.

23. A method for producing a sample containing a diastereomeric excess of Compound M-2, the method comprising heating a mixture having a first ratio of Compound P-2 N-Boc-D-phenylalanine salt to Compound M-2 N-Boc-D-phenylalanine salt to obtain a mixture having a second ratio of Compound P-2 N-Boc-D-phenylalanine salt to Compound M-2 N-Boc-D-phenylalanine salt.

24. (canceled)

25. (canceled)

26. A compound which is
the N-Boc-D-phenylalanine salt of (2M)-2-(4-(4-(aminomethyl)-1-oxo-1,2-dihydrophthalazin-6-yl)-1-methyl-1H-pyrazol-5-yl)-3-fluoro-3,4-dihydronaphthalene-1-carbonitrile;
the N-Boc-D-phenylalanine salt of (2P)-2-(4-(4-(aminomethyl)-1-oxo-1,2-dihydrophthalazin-6-yl)-1-methyl-1H-pyrazol-5-yl)-3-fluoro-3,4-dihydronaphthalene-1-carbonitrile;
the N-Boc-L-phenylalanine salt of (2M)-2-(4-(4-(aminomethyl)-1-oxo-1,2-dihydrophthalazin-6-yl)-1-methyl-1H-pyrazol-5-yl)-3-fluoro-3,4-dihydronaphthalene-1-carbonitrile;
the N-Boc-L-phenylalanine salt of (2P)-2-(4-(4-(aminomethyl)-1-oxo-1,2-dihydrophthalazin-6-yl)-1-methyl-1H-pyrazol-5-yl)-3-fluoro-3,4-dihydronaphthalene-1-carbonitrile;
the N-Boc-D-phenylalanine salt of (2M)-2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile;
the N-Boc-D-phenylalanine salt of (2P)-2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile;
the N-Boc-L-phenylalanine salt of (2M)-2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile; or
the N-Boc-L-phenylalanine salt of (2P)-2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile.

27-33. (canceled)

34. A crystalline form of N-Boc-D-phenylalanine salt of (2M)-2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile.

35. Crystalline Form A of (2M)-2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile: Crystalline Form A of (2M)-2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloric acid salt; or Crystalline Form B of (2M)-2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile hydrochloric acid salt.

36. (canceled)

37. (canceled)

38. A method of separating a mixture of atropisomers, the method comprising:
(a) forming a mixture of a solvent, a first atropisomer and a second atropisomer in a crystallization vessel;
(b) producing a liquid phase and a solid phase by, if necessary, adjusting the temperature of the mixture to a temperature in which the first atropisomer and second atropisomer have different solubilities, where the solid phase contains an enantiomeric excess of the first atropisomer and the liquid phase contains an enantiomeric excess of the second atropisomer;
(c) removing a portion of the liquid phase;
(d) subjecting the removed liquid phase portion to conditions sufficient to produce an increased amount of the first atropisomer in the removed liquid phase relative to the amount in the liquid phase in (b);
(e) returning the removed liquid phase produced in (d) to the crystallization vessel; and
(f) isolating crystals of the first atropisomer.

39. A method of separating a mixture of atropisomers according to claim 38, wherein the atropisomers are of a compound having the formula 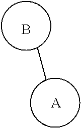
wherein
the A-ring is 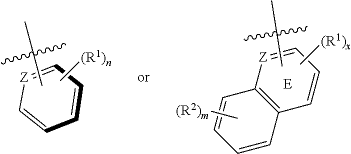
wherein the point of attachment of the bicyclic A-ring to the B-ring is on ring E;
the B-ring is a 6-10 membered aryl group, 5-10 membered heteroaryl group, 5-10 membered heterocycloalkyl group, an amide group, a sulfone group, a sulfoxide group, an olefin group, an amine group, or an ether group, each of which is optionally substituted;
Z is N, CH or CR¹;
n is 0, 1, 2, 3, or 4;
x is 0, 1, or 2;
m is 0, 1, 2, 3 or 4;
each R¹ is independently C₁-C₆ alkyl, C₁-C₆ alkoxy, C₃-C₈ cycloalkyl, C₃-C₈ cycloalkyloxy, halogen, cyano, hydroxy, amino, or mono- or di-C₁-C₆ alkyl amino; and
each R² is independently C₁-C₆ alkyl, C₁-C₆ alkoxy, C₃-C₈ cycloalkyl, C₃-C₈ cycloalkyloxy, halogen, cyano, hydroxy, amino, or mono- or di-C₁-C₆ alkyl amino;
provided that at least one of the positions on the A-ring ortho to the point of attachment of the A-ring to the B-ring is substituted with R¹.

40. (canceled)

41. (canceled)

42. A method of separating a mixture of atropisomers according to claim 38, wherein the first atropisomer is (2M)-2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile (Compound M-2) and the second atropisomer is (2P)-2-(4-(4-(aminomethyl)-1-oxo-1,2-dihydrophthalazin-6-yl)-1-methyl-1H-pyrazol-5-yl)-4-chloro-6-cyclopropoxy-3-fluorobenzonitrile (Compound P-2).

43-47. (canceled)

48. A method of separating a mixture of atropisomers according to claim 1, wherein the subjecting in (d) comprises heating at a temperature of from about 80-200° C.

49. A method according to claim 1, wherein
the contacting generates a slurry comprising
a solid phase enriched with the Compound M-2 N-Boc-D-phenylalanine salt, and
a liquid phase enriched with the Compound P-2 N-Boc-D-phenylalanine salt, and
the method further comprises
separating a portion of the liquid phase from the slurry, and
heating the liquid phase to produce a heated mixture having a ratio of Compound P-2 N-Boc-D-phenylalanine salt to Compound M-2 N-Boc-D-phenylalanine salt that is different than the ratio prior to the heating.

50. (canceled)

51. (canceled)

52. (canceled)

53. A system for separating atropisomers comprising a crystallization module, an epimerization module and an optional collection module.

54. (canceled)

55. (canceled)

56. A method for separating atropisomers comprising
selectively crystallizing a less soluble atropisomer in a crystallization module; and
epimerizing a more soluble atropisomer in an epimerization module;
wherein soluble material is continuously or semi-continuously provided from the crystallization module directly or indirectly to the epimerization module, and material from the epimerization module is at least semi-continuously returned to the crystallization module.

57. (canceled)

58. (canceled)

59. (canceled)
